(19) United States
(12) Patent Application Publication
(10) Pub. No.: US 2004/0199241 A1
(43) Pub. Date: Oct. 7, 2004

(54) Silk stent grafts

(75) Inventors: David M. Gravett, Vancouver (CA); Pierre E. Signore, Vancouver (CA); Kaiyue Wang, Vancouver (CA); Philip M. Toleikis, Vancouver (CA); Dechi Guan, Vancouver (CA); Zengxuan Hu, Vancouver (CA); Arpita Maiti, Vancouver (CA)

Correspondence Address: SEED INTELLECTUAL PROPERTY LAW GROUP PLLC, 701 FIFTH AVENYUE, SUITE 6300, SEATTLE, WA, 98104-7092, US

(73) Assignee: Angiotech International AG, Zug (CH)

(21) Appl. No.: 10/748,747

(22) Filed: Dec. 29, 2003

Related U.S. Application Data
(60) Provisional application No. 60/437,463, filed on Dec. 30, 2002.

(51) Int. Cl. A61F 2/06

(52) U.S. Cl. 623/1.13; 623/901

(57) ABSTRACT

Silk-containing stent grafts are provided comprising an endoluminal stent and a graft, wherein the silk is induces the in vivo adhesion of the stent graft to vessel walls, or, otherwise induces or accelerates an in vivo fibrotic reaction causing said stent graft to adhere to vessel wall. Also provided are methods for making and using such stent grafts.

### CROSS REFERENCE TO RELATED APPLICATIONS

This application claims the benefit of U.S. Provisional Patent Application No. 60/437,463, filed Dec. 30, 2002, where this provisional application is incorporated herein by reference in its entirety.

### FIELD OF THE INVENTION

The present invention relates generally to pharmaceutical compositions, methods and devices, more specifically to stent grafts, and particularly to stent grafts that contain silk and methods for making and using such stent grafts.

### BACKGROUND OF THE INVENTION

Stent grafts are utilized not only to hold open a passageway, but also to bridge across diseased vasculature from healthy vessel to healthy vessel. A common application of stent grafts is to bypass an abdominal aortic aneurysm (AAA). Briefly, a stent graft is inserted over a guide wire, from the femoral or iliac artery, and deployed within the aneurysm, resulting in maintenance of blood flow from an aorta of acceptable (usually normal) caliber above the aneurysm to a portion of aorta or iliac artery(s) of acceptable (usually normal) caliber below the aneurysm. Blood flow is thereby excluded from entering the aneurysm sac. Blood within this excluded sac thromboses and the aneurysm thus has no flow within it, presumably reducing the pressure and thus its tendency to burst.

While generally useful, presently available stent grafts have a number of shortcomings. For example, current stent grafts are prone to persistent leakage around the area of the stent graft. Hence, pressure within the aneurysm sac stays at or near arterial pressure, and there remains a risk that the sac will rupture. There are three common types of perigraft leakage. The first type is direct leakage around the stent graft. This can be persistent from the time of insertion because of poor sealing between the stent graft and vessel wall, or can develop later because the seal is lost. In addition, this problem can develop due to changes in the position or orientation of the stent graft in relation to the aneurysm as the aneurysm grows, shrinks, elongates or shortens with time after treatment. The second type of perigraft leak can occur because there are side arteries extending out from the treated segment of blood vessel. Once the device excludes the aneurysm, flow can reverse within these blood vessels and continue to fill the aneurysm sac around the stent graft. The third type of perigraft leak can occur because of disarticulation of the device (in the case of modular devices) or because of the development of holes within the graft material. The continuous pulsation of the vessel can cause the graft material to rub against a metallic stent tyne, leading to hole formation and eventually causing graft failure. In addition, disarticulation of the device can develop due to changes in shape of the aneurysm as it grows, shrinks, elongates or shortens with time after treatment.

Stent grafts are also limited in their application to only selected patients with aneurysms. For example, endovascular stents are an advance in the treatment of AAA as they offer the avoidance of standard therapy, which is a major operation with a significant morbidity, mortality, long hospital stays, and prolonged recovery time. However, endovascular technology is only applicable to certain patients with AAA because of (a) lack of a suitable route of access via the blood vessels to the intended site of deployment which prevents insertion of the device and (b) the patient's anatomy.

In order to effectively exclude an aneurysm, the graft material needs to be of a certain strength and durability, or else it will tear. Typically, in order to achieve these properties, a polyester (e.g., polyester sold, e.g., under the trade name DACRON (E. I. DuPont De Nemours and Company, Wilmington, Del.) or poly(tetrafluoroethylene) (PTFE)) graft material of conventional “surgical” thickness may be used. This level of thickness is needed in order to convey adequate strength to the material. The thickness of the material results in the need for delivery devices typically of 24 to 27 French (8 to 9 millimeter diameter) and occasionally up to 32 French. This requires surgical exposure of the insertion site, usually a common femoral artery, and limits the application of the technology, as a larger delivery device is more difficult to manipulate through the iliac artery to the intended site of delivery. Even “low profile” devices, which use thinner graft material, are of a sufficient size that a surgical exposure of the blood vessel through which the device is inserted is required. If the iliac arteries or aorta are very tortuous, (as is frequently the case in AAA), or heavily calcified and diseased (another frequent association with AAA), this may be a contraindication to treatment, or cause of failure of attempted treatment, because of inability to advance a device to the site of deployment or potential for iliac artery rupture.

A stent graft is typically used to bridge a diseased artery (usually an aneurysm), extending from a portion of artery of acceptable caliber above the diseased region to an artery of acceptable caliber below the diseased region. To achieve a long lasting seal, the artery of acceptable caliber above the diseased region (“proximal neck”) should be at least 1.5 cm long without a major branch vessel arising from it. The artery of acceptable caliber below the diseased region (“distal neck”) should be at least 1.0 cm long without a major branch vessel arising within that 1 cm length of vessel. Shorter “necks” at either end of the diseased segment, necks which are sloping rather than cylindrical, or necks which are smaller than the aneurysm but still dilated in comparison to the normal diameter for a vessel in this location predispose to failure of sealing around the stent graft or delayed perigraft leaks. One further difficulty with present stent grafts is that over time certain devices have a tendency to migrate distally within the abdominal aorta. Such migration results in device failure, perigraft leak and vessel occlusion.

The present invention provides a stent graft that overcomes problems associated with existing stent grafts.

### BRIEF SUMMARY OF THE INVENTION

Briefly stated, the present invention provides silk-containing stent grafts, compositions for modifying or coating stent grafts with silk, and methods for making and using these grafts.

Within one aspect of the invention, a stent graft is provided that includes an endoluminal stent and a graft, wherein the stent graft includes silk. The silk induces a response in a host who receives the stent graft, where the response can lead to enhanced adhesion between the silk stent graft and the host's tissue that is adjacent to the silk of the silk stent graft. In various aspects, the silk comprises fibroin and/or sericin. The silk may be natural, unmodified silk, or it may be chemically modified silk, e.g., acylated silk. However, the silk should not be modified to such an extent that it eliminates the ability of the silk to induce the host to generate a biological response that can increase adhesion between the stent graft and the tissue in the host that is adjacent to the silk of the silk stent graft. The silk may be from any of various sources, e.g., from a silkworm or from a spider, or from recombinant sources. The silk may be attached to the graft by any of various means, e.g., by interweaving the silk into the graft or by adhering the silk to the graft (e.g., by means of an adhesive or by means of suture). The silk may be in the form of a thread, a braid, a sheet, powder, etc. As for the location of the silk on the stent graft, in one aspect, the silk may be attached only the exterior of the stent, and/or in another aspect the silk may be attached to distal regions of the stent graft, in order to assist in securing those distal regions to neighboring tissue in the host. In one aspect, a plurality of separated silk braids is attached to the stent graft. The silk may be attached to the stent portion of the stent graft and/or to the graft portion of the stent graft.

A wide variety of stent grafts may be utilized within the context of the present invention, depending on the site and nature of treatment desired. Stent grafts may be, for example, bifurcated or tube grafts, cylindrical or tapered, self-expandable or balloon-expandable, unibody or, modular, etc.

In addition to silk, the stent graft of the present invention may contain a coating on some or all of the silk, where the coating degrades upon insertion of the stent graft into a host, the coating thereby delaying contact between the silk and the host. Suitable coatings include, without limitation, gelatin, degradable polyesters (e.g., PLGA, PLA, MePEG-PLGA, PLGA-PEG-PLGA, and copolymers and blends thereof), cellulose and cellulose derivatives (e.g., hydroxypropyl cellulose), polysaccharides (e.g., hyaluronic acid, dextran, dextran sulfate, chitosan), lipids, fatty acids, sugar esters, nucleic acid esters, polyanhydrides, polyorthoesters and polyvinylalcohol (PVA).

The silk-containing stent grafts of the present invention may, in one aspect, contain a biologically active agent, where the agent is released from the stent graft and then induces an enhanced cellular response (e.g., cellular or extracellular matrix deposition) and/or fibrotic response in a host into which the stent graft has been inserted. Exemplary agents include, without limitation, bleomycin or an analogue or derivative thereof, talcum powder, talc, ethanol, metallic beryllium and oxides thereof, silver nitrate, copper, silk, silica, crystalline silicates, quartz dust, and vinyl chloride. Exemplary polymeric agents include poly(ethylene-co-vinylacetate), polyurethane, polymers and copolymers of acrylic acid, and polymers of vinyl chloride. The agent may be an adhesive, such as, cyanoacrylate, crosslinked poly(ethylene glycol)—methylated collagen, and derivatives thereof; a protein, carbohydrate or peptide that contains cellular adhesion sequences; an inflammatory cytokine (e.g., TGFβ, PDGF, VEGF, aFGF, bFGF, TNFα, NGF, GM-CSF, IGF-a, IL-1, IL-8, IL-6, growth hormone, EDGF, CTGF, and peptide and non-peptide agonists, analogues and derivatives thereof); a component of extracellular matrix (e.g., vitronectin, fibronectin, chondroitin sulphate, laminin, hyaluronic acid, elastin, fibrin, fibrinogen, bitronectin, proteins found in basement membrane, fibrosin, or collagen); polylysine, chitosan, or N-carboxybutylchitosan; a factor produced by immune cells (e.g., Interleukin-2 (IL-2), Interleukin-4 (IL-4), Interleukin-1 (IL-1), Interleukin-8 (IL-8), Interleukin-6 (IL-6) and peptide and non-peptide agonists, analogues and derivatives thereof, Granulocyte-Monocyte Colony-Stimulating-Factor (GM-CSM), monocyte chemotactic protein, histamine, and cell adhesion molecules; naturally occurring and synthetic peptides containing the RGD residue sequence; bone morphogenic protein (BMP) (e.g., BMP-2, BMP-3, BMP-4, BMP-5, BMP-6, or BMP-7); an inorganic and organic small anionic molecule stimulant; and DNA and RNA sequences which are capable of promoting synthesis of proteins that stimulate cell growth.

In one aspect, the stent graft of invention further comprises a proliferative agent that stimulates cellular proliferation. Representative examples of proliferative agents include dexamethasone, isotretinoin, 17-β-estradiol, diethylstibesterol, cyclosporin A, all-trans retinoic acid (ATRA), and analogues and derivatives thereof.

In another aspect, the stent graft of the invention further comprises a biologically active agent that inhibits or prevents expansion of an aneurysm, such as a caspase inhibitor (e.g., VX-799); an MMP inhibitor (e.g., BATIMASTAT or MARIMISTAT); a tissue inhibitor of matrix metalloproteinases (TIMP); a cytokine inhibitor (e.g., chlorpromazine, mycophenolic acid, rapamycin, or 1α-hydroxy vitamin D₃); a MCP-1 antagonist (e.g., nitronaproxen, Bindarit, or 1-alpha-25 dihydroxy vitamin D₃); a TNFa antagonist or a TACE inhibitor (e.g., E-5531, AZD-4717, glycophosphopeptical, UR-12715, cilomilast, infliximab, lentinan, or etanercept); an IL-1, ICE, and IRAK antagonist (e.g., E-5090, CH-172, CH-490, AMG-719, iguratimod, AV94-88, pralnacasan, ESONARIMOD, or tranexamic acid); a chemokine receptor antagonist (e.g., ONO-4128, L-381, CT-112, AS-900004, SCH-C, ZK-811752, PD-172084, UK-427857, SB-380732, vMIP II, SB-265610, DPC-168, TAK-779, TAK-220, or KRH-1120); or an anti-inflammatory agent (e.g., dexamethasone, cortisone, fludrocortisone, prednisone, prednisolone, 6α-methylprednisolone, triamcinolone, betamethasone, and analogues and derivatives thereof).

In addition, the present invention provides methods for forming a silk-containing stent graft. In various aspects, which are exemplary only, the silk may be attached to the stent graft by interweaving the silk into the graft, or the silk may be attached to the stent graft by means of an adhesive, or the silk may be attached to the stent graft by means of suture. In one aspect the silk is attached only to the outside of the stent graft, and/or the silk may be attached to distal regions of the stent graft. In one aspect, the silk is added to the stent graft in an amount effective to induce a biological response in a host into which the stent graft has been inserted, where the biological response is a cellular matrix deposition between the stent graft and tissue adjacent to the stent graft. In a related aspect, the silk is added to the stent graft in an amount effective to induce a biological response in a host into which the stent graft has been inserted, where the biological response is a cellular or extracellular matrix deposition between the stent graft and tissue adjacent to the stent graft. Optionally, the presence of the silk induces an enhanced biological response, i.e., a greater biological response than would have occurred in the absence of the silk on the stent graft.

Also provided by the present invention are methods for treating patients having aneurysms (e.g., abdominal, thoracic, or iliac aortic aneurysms), for bypassing a diseased portion of a vessel, or for creating communication or a passageway between one vessel and another (e.g., artery to vein or vice versa, or artery to artery or vein to vein), such that risk of rupture of the aneurysm is reduced. In one embodiment, the stent graft is delivered into a patient (e.g., by balloon catheter) in a constrained form, and self-expands into place after release of a constraining device. The methods utilize the silk-containing stent grafts of the present invention. As utilized herein, it should be understood that “reduction in the risk of rupture” or “prevention of the risk of rupture” refers to a statistically significant reduction in the, number, timing, or, rate of rupture, and not to a permanent prohibition of any rupture. Likewise, a “reduction in the risk of perigraft leakage refers to statistically significant enhancement in the effectiveness and/or effective lifetime of a stent graft, and not to a permanent or complete cessation of perigraft leakage.

The present invention addresses shortcomings in current stent graft technology by providing novel compositions, methods for preparing, and devices related to silk-containiing stent grafts. The invention further provides other related advantages as disclosed below.

These and other aspects of the present invention will become evident upon reference to the following detailed description and attached drawings. In addition, various references are set forth herein which describe in more detail certain procedures and/or compositions (e.g., polymers), and these references are incorporated by reference in their entirety.

### BRIEF DESCRIPTION OF THE DRAWINGS

FIG. 1 is a schematic illustration of a representative stent graft. Dashed lines indicate coating of the graft with a desired agent at each end of the graft.

FIG. 2 is a cross-sectional view of the stent graft illustrated in FIG. 1.

FIG. 3 is a schematic illustration of a silk stent graft of the present invention having silk sutures that are secured to the stent graft in a horizontal, diagonal or vertical manner.

FIG. 4 is a schematic illustration of a silk stent graft of the present invention having silk sutures that are attached at either one end or both ends of the silk threads, where the silk extends some distance from the stent graft.

FIG. 5 is a graph showing the % activation of proliferation in smooth muscle cells as a function of cyclosporin A concentration.

FIG. 6 is a bar graph showing the average number of cells migrating for untreated and paclitaxel treated primary smooth muscle cells in response to rhPDDF-BB.

FIG. 7 is a bar graph showing the area of granulation tissue in carotid arteries exposed to silk coated perivascular PU films relative to arteries exposed to uncoated PU films.

FIG. 8 is a bar graph showing the area of granulation tissue in carotid arteries exposed to silk suture coated perivascular PU films relative to arteries exposed to uncoated PU films.

FIG. 9 is a bar graph showing the area of granulation tissue in carotid arteries exposed to natural and purified silk powder and wrapped with perivascular PU film relative to a control group in which arteries are wrapped with perivascular PU film only.

FIG. 10 is a bar graph showing the area of granulation tissue (at 1 month and 3 months) in carotid arteries sprinkled with talcum powder and wrapped with perivascular PU film relative to a control group in which arteries are wrapped with perivascular PU film only.

FIG. 11 is a photograph (100×) showing the cross section of a carotid artery one month after insertion of a stent graft (control).

FIG. 12 is a photograph (100×) showing the cross section of a carotid artery one month after insertion of a silk covered stent graft.

### DETAILED DESCRIPTION OF THE INVENTION

### Definitions

Prior to setting forth the invention, it may be helpful to an understanding thereof to first set forth definitions of certain terms that are used hereinafter.

“Stent graft” refers to devices comprising a graft or wrap (composed of a textile, polymer, or other suitable material such as biological tissue) which maintains the flow of fluids (e.g., blood) from one portion of a vessel to another, and an endovascular scaffolding or stent (including expandable and inflatable stent structures) that holds open a body passageway and/or supports the graft or wrap. The graft or wrap may be woven within a stent, contained within the lumen of a stent, and/or be located exterior to a stent.

“Fibrosis” or “Scarring” refers to the formation of fibrous tissue in response to injury or medical intervention. Therapeutic agents which promote fibrosis or scarring (also referred to herein as fibrosing or fibrosis inducing agents) can do so through one or more mechanisms including: inducing or promoting angiogenesis, stimulating migration or proliferation of connective tissue cells (such as fibroblasts, and/or smooth muscle cells), inducing ECM (extracellular matrix) production, and/or promoting tissue remodeling. In addition, numerous therapeutic agents described in this invention will have the additional benefit of also promoting tissue regeneration (the replacement of injured cells by cells of the same type).

Silk refers to a fibrous protein, and may be obtained from a number of sources, typically spiders and silkworms. Typical silks contain about 75% of actual fiber, referred to as fibroin, and about 25% sericin which is a gummy protein that holds the filaments together. Silk filaments are generally very fine and long—as much as 300-900 meters long. There are several species of domesticated silkworm that are used in commercial silk production, however, Bombyx mori is the most common, and most silk comes from this source. Other suitable silkworms include Philosamia cynthia ricini, Antheraea yamamai, Antheraea pernyi, and Antheraea mylitta. The silk can be processed to produce the raw silk or floss silk. Some of these processes involve degumming the silk. The steps to produce the different types of silk can include steps that can remove some or all of the sericin. Spider silk is relatively more difficult to obtain, however, recombinant techniques hold promise as a means to obtain spider silk at economical prices (see, e.g., U.S. Pat. Nos. 6,268,169; 5,994,099; 5,989,894; and 5,728,810, which are exemplary only). Biotechnology has allowed researchers to develop other sources for silk production, including animals (e.g., goats) and vegetables (e.g., potatoes). Silk from any of these sources may be used in the present invention, however, in one aspect of the invention the silk is not exclusively spider-derived silk or a genetically engineered spider silk as disclosed in, e.g., U.S. patent application No. US2001/0053931 A1. In one aspect of the present invention, the silk is not exclusively biological or genetically-engineered spider silk or a derivative thereof, such as spider silk derived from Nephila clavipes, or a genetically engineered copy or variant thereof. In another aspect of the invention, the stent graft does not include any spider silk. In another aspect, less than 50% of the silk present in a stent graft of the present invention is biologically or genetically-engineered spider silk or a derivative thereof.

Raw silk is typically twisted into a strand sufficiently strong for weaving or knitting. Four different types of silk thread may be produced by this procedure: organzine, crepe, tram and thrown singles. Organzine is a thread made by giving the raw silk a preliminary twist in one direction and then twisting two of these threads together in the opposite direction. Crepe is similar to organzine but is twisted to a much greater extent. Twisting in only one direction two or more raw silk threads makes tram. Thrown singles are individual raw silk threads that are twisted in only one direction. Any of these types of silk threads may be used in the present invention.

The silk can be used in the form of threads, monofilament yarn, multifilament yarn, braids, powders as well as oligomers of the silk protein.

In addition to raw silk, commercially available silk sutures that are used for surgical closure applications also can be used in the present invention. Examples of such commercially available silk sutures include, but are not limited to, those sold by Ethicon Inc. (Somerville, N.J.), USSC/David&Geck/Tyco (Norwalk, Conn.) and Suru International (India).

In addition to silk threads, fibers and yarns, silk in other forms can be used. A commercially available silk protein is available from Croda, Inc., of Parsippany, N.J., and is sold under the trade names CROSILK LIQUID (silk amino acids), CROSILK 10,000 (hydrolyzed silk), CROSILK POWDER (powdered silk), and CROSILKQUAT (cocodiammonium hydroxypropyl silk amino acid). Another example of a commercially available silk protein is SERICIN, available from Pentapharm, LTD, a division of Kordia, BV, of the Netherlands. Further details of such silk protein mixtures can be found in U.S. Pat. No. 4,906,460, to Kim, et al., assigned to Sorenco. Silk useful in the present invention includes natural (raw) silk, hydrolyzed silk, and modified silk, i.e., silk that has undergone a chemical, mechanical, or vapor treatment, e.g., acid treatment or acylation (see, e.g., U.S. Pat. No. 5,747,015). However, as mentioned above, in one aspect of the invention the silk is not spider-derived silk or genetically engineered spider silk. In a further optional aspect, the stent graft of the present invention contains silk that induces a greater tissue inflammatory response than does spider silk. In yet another optional embodiment, the silk present in the stent graft of the present invention promotes a tissue inflammatory response.

The silk used in the present invention may be in any suitable form that allows the silk to be joined (e.g., physically, mechanically, chemically or via coating) with the stent graft, e.g., the silk may be in thread or powder-based forms. Generally, the silk is not released from the stent graft after insertion into the patient, however, in certain applications, it may be desirable that the silk be released from the stent graft.

Furthermore, the silk may have any molecular weight. This molecular weight can range from what is naturally found to molecular weights that can typically be obtained by the hydrolysis of natural silk, where the extent and harshness of the hydrolysis conditions determines the product molecular weight. For example, silk powders can have a molecular weight of about 100,000 to 300,000 Da while a soluble silk may have an average (number or weight) molecular weight of 200 to 5,000. See, e.g., JP-B-59-29199 (examined Japanese patent publication) for a description of conditions that may be used to hydrolyze silk.

A discussion of silk may be found in the following documents, which are exemplary only: Hinman, M. B., et al. “Synthetic spider silk: a modular fibre” Trends in Biotechnology, 2000, 18(9) 374-379; Vollrath, F. and Knight, D. P. “Liquid crystalline spinning of spider silk” Nature, 2001, 410(6828) 541-548; and Hayashi, C. Y., et al. “Hypotheses that correlate the sequence, structure, and mechanical properties of spider silk proteins” Int. J. Biol. Macromolecules, 1999, 24(2-3), 265-270; and U.S. Pat. No. 6,427,933.

The silk utilized in the present invention is intended to cause or induce a biological reaction by the host who has received the stent graft. In one aspect, the silk is utilized in order to induce a fibrotic reaction so that scarring occurs in the vicinity of the stent graft. To this extent then, the silk is non-biocompatible.

As discussed above, the present invention provides compositions, methods and devices relating to silk-containing stent grafts, where the presence of silk greatly increases the success and application of the stent graft. Described in more detail below are methods for constructing silk-containing stent grafts, compositions and methods for generating silk-containing stent grafts that adhere to a vessel wall, and methods for utilizing such stent grafts.

### Stent Grafts

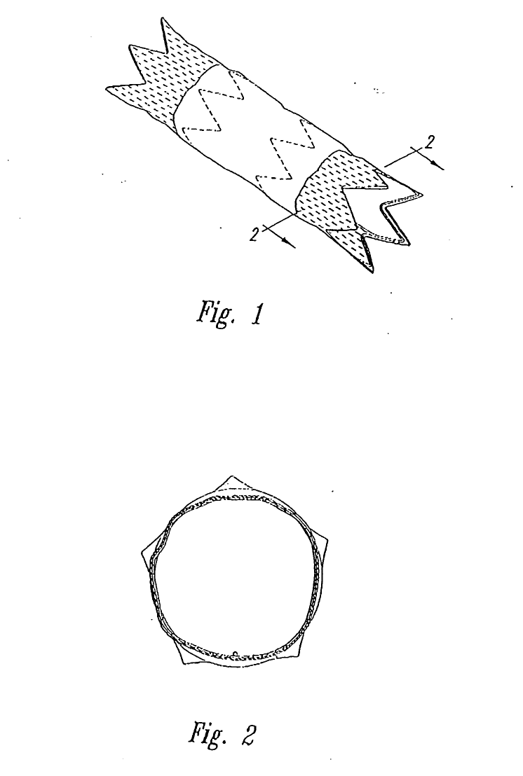

As noted above, stent grafts are devices that include a graft or wrap which maintains the flow of fluids (e.g., blood) from one portion of a vessel to another, or from one blood vessel to another, and an endovascular scaffolding or stent which holds open a body passageway and/or supports the graft or wrap. One representative stent graft is illustrated in FIGS. 1 and 2.

The graft portion of the stent may be composed of a textile, polymer, or other suitable material such as biological tissue. Representative examples of suitable graft materials include textiles (including, e.g., woven and non-woven materials) made from polymeric fibers. Polymeric fibers for use in textiles may be formed from a variety of polymers, including, for example, nylon, acrylonitrile polymers and copolymers (available, e.g., under the trade name ORLON (E. I. DuPont De Nemours and Company, Wilmington, Del.)), polyesters (available, e.g., under the trade name DACRON (E. I. DuPont De Nemours and Company)), and poly(tetrafluoroethylene) (available, e.g., under the trade name TEFLON (E. I. DuPont De Nemours and Company)). Other representative examples of graft materials include non-textiles, such as expanded polytetrafluroethylene (ePTFE). The graft or wrap may be woven within a stent, contained within the lumen of a stent and/or be located exterior to a stent.

Representative examples of stent grafts, and methods for making and utilizing such grafts are described in more detail in U.S. Pat. No. 5,810,870 entitled “Intraluminal Stent Graft”; U.S. Pat. No. 5,776,180 entitled “Bifurcated Endoluminal Prosthesis”; U.S. Pat. No. 5,755,774 entitled “Bistable Luminal Graft Endoprosthesis”; U.S. Pat. Nos. 5,735,892 and 5,700,285 entitled “Intraluminal Stent Graft”; U.S. Pat. No. 5,723,004 entitled “Expandable Supportive Endoluminal Grafts”; U.S. Pat. No. 5,718,973 entitled “Tubular Intraluminal Graft”; U.S. Pat No. 5,716,365 entitled “Bifurcated Endoluminal Prosthesis”; U.S. Pat. No. 5,713,917 entitled “Apparatus and Method for Engrafting a Blood Vessel”; U.S. Pat. No. 5,693,087 entitled “Method for Repairing an Abdominal Aortic Aneurysm”; U.S. Pat. No. 5,683,452 entitled “Method for Repairing an Abdominal Aortic Aneurysm”; U.S. Pat. No. 5,683,448 entitled “Intraluminal Stent and Graft”; U.S. Pat. No. 5,653,747 entitled “Luminal Graft Endoprosthesis and Manufacture Thereof”; U.S. Pat. No. 5,643,208 entitled “Balloon Device of Use in Repairing an Abdominal Aortic Aneurysm”; U.S. Pat. No. 5,639,278 entitled “Expandable Supportive Bifurcated Endoluminal Grafts”; U.S. Pat. No. 5,632,772 entitled “Expandable Supportive Branched Endoluminal Grafts”; U.S. Pat. No. 5,628,788 entitled “Self-Expanding Endoluminal Stent-Graft”; U.S. Pat. No. 5,591,229 entitled “Aortic Graft for Repairing an Abdominal Aortic Aneurysm”; U.S. Pat. No. 5,591,195 entitled “Apparatus and Methods for Engrafting a Blood Vessel”; U.S. Pat. No. 5,578,072 entitled “Aortic Graft and Apparatus for Repairing an Abdominal Aortic Aneurysm”; U.S. Pat. No. 5,578,071 entitled “Aortic Graft”; U.S. Pat. No. 5,571,173 entitled “Graft to Repair a Body Passageway”; U.S. Pat. No. 5,571,171 entitled “Method for Repairing an Artery in a Body”; U.S. Pat. No. 5,522,880 entitled “Method for Repairing an Abdominal Aortic Aneurysm”; U.S. Pat. No. 5,405,377 entitled “Intraluminal Stent”; U.S. Pat. No. 5,360,443 entitled “Aortic Graft for Repairing an Abdominal Aortic Aneurysm”; U.S. Pat. No. 6,488,701 entitled “Stent-graft assembly with thin-walled graft component and method of manufacture”; U.S. Pat. No. 6,482,227 entitled “Stent graft having improved attachment within a body vessel”; U.S. Pat. No. 6,458,152 entitled “Coiled sheet graft for single and bifurcated lumens and methods of making and use”; U.S. Pat. No. 6,451,050 entitled “Stent graft and method”; U.S. Pat. No. 6,395,018 entitled “Endovascular graft and process for bridging a defect in a main vessel near one of more branch vessels”; U.S. Pat. No. 6,390,098 entitled “Percutaneous bypass with branching vessel”; U.S. Pat. No. 6,361,637 entitled “Method of making a kink resistant stent-graft”; U.S. Pat. No. 6,348,066 entitled “Modular endoluminal stent-grafts and methods for their use”; U.S. Pat. No. 6,344,054 entitled “Endoluminal prosthesis comprising stent and overlying graft cover, and system and method for deployment thereof; U.S. Pat. No. 6,325,820 entitled “Coiled-sheet stent-graft with exo-skeleton”; U.S. Pat. No. 6,322,585 entitled “Coiled-sheet stent-graft with slidable exo-skeleton”; U.S. Pat. No. 6,319,278 entitled “Low profile device for the treatment of vascular abnormalities”; U.S. Pat. No. 6,296,661 entitled “Self-expanding stent-graft”; U.S. Pat. No. 6,245,100 entitled “Method for making a self-expanding stent-graft”; U.S. Pat. No. 6,238,432 entitled “Stent graft device for treating abdominal aortic aneurysms”; U.S. Pat. No. 6,214,039 entitled “Covered endoluminal stent and method of assembly”; U.S. Pat. No. 6,168,610 entitled “Method for endoluminally excluding an aortic aneurysm”; U.S. Pat. No. 6,165,213 entitled “System and method for assembling an endoluminal prosthesis”; U.S. Pat. No. 6,165,210 entitled “Self-expandable helical intravascular stent and stent-graft”; U.S. Pat. No. 6,143,022 entitled “Stent-graft assembly with dual configuration graft component and method of manufacture”; U.S. Pat. No. 6,123,722 entitled “Stitched stent grafts and methods for their fabrication”; U.S. Pat. No. 6,117,167 entitled “Endoluminal prosthesis and system for joining”; U.S. Pat. No. 6,099,559 entitled “Endoluminal support assembly with capped ends”; U.S. Pat. No. 6,042,605 entitled “Kink resistant stent-graft”; U.S. Pat. No. 6,015,431 entitled “Endolumenal stent-graft with leak-resistant seal”; U.S. Pat. No. 5,957,974 entitled “Stent graft with braided polymeric sleeve”; U.S. Pat. No. 5,916,264 entitled “Stent graft”; U.S. Pat. No. 5,906,641 entitled “Bifurcated stent graft”; U.S. Pat. No. 5,891,191 entitled “Cobalt-chromium-molybdenum alloy stent and stent-graft”; U.S. Pat. No. 5,824,037 entitled “Modular intraluminal prostheses construction and methods”; U.S. Pat. No. 5,824,036 entitled “Stent for intraluminal grafts and device and methods for delivering and assembling same”; U.S. Publication Nos. 2003/0120331; 2003/120338; and 2003/0125797; U.S. Pat. No. 6,334,867, and PCT Publication No. WO 99/37242.

### Silk Stent Grafts

In one aspect, the present invention provides a stent graft to which silk has been secured. The basic stent graft may be any of the stent grafts described previously, or any other similar stent graft. The silk that is present on the stent graft induces an enhanced fibrotic response between the stent graft and the tissue adjacent to the in vivo stent graft. Thus, in one aspect, the silk has the feature that it will induce an inflammatory response when contacted with a mammal. In another aspect, the silk has the feature that it will induce a cellular and/or extracellular matrix deposition response in an animal that is contacted with the silk. That is, absent the silk, the stent graft would generate a “normal” adhesion between the adjacent tissue and the stent graft, while in the presence of the silk the same stent/graft is capable of generating an enhanced adhesion via, e.g., an enhanced matrix deposition response to the presence of the silk. In one aspect of the invention, the silk excludes silks that do not induce an enhanced fibrotic response.

While the silk may be in any form or shape, e.g., sheet, powder, thread, braid, filament, fiber, film, foam, and the like. In certain embodiments, the silk is in the form of a thread or powder. While the following discussion is primarily in terms of threads, the same principles and teachings apply to other forms and shapes of the silk.

The silk-containing threads will typically range in size from 1 nm to 3 mm in diameter although other sizes may be used and will also be effective. The threads can be individual thread (a monofilament), a multitude of threads (multifilament yarn), a braid, a knitted thread or a woven thread. The threads can be used “as is”, or they can be further processed into a knitted or woven material that is then attached to the stent graft. The threads can be made such that there are fiber(s) that protrude from the thread. These protruding fibers will further increase the exposed surface area, thereby enhancing the biological response when the stent graft is inserted into a host. The fibers that protrude from the thread can be of the same composition as the thread material or they can comprise a different composition than the thread material.

As discussed in further detail below, the silk may be secured to the stent graft by any of a number of methods. Suitable methods include, without limitation, interweaving the silk into the graft, interweaving the silk into the stent structure;

attaching the silk to the stent via knotting or suturing it around the stent structure;

attaching the silk to the stent graft by means of an adhesive; and using one or more sutures to “sew” the silk onto the stent graft. In one aspect, a plurality of separated silk braids or threads is attached to the stent graft.

The silk itself may be natural silk, as obtained from, e.g., silkworms or spiders. Alternatively, the silk may be a recombinant silk, or a chemically modified silk (e.g., acylated silk). In another aspect, the silk can be commercially available silk sutures. In one aspect, the silk includes fibroin, which is a component of natural silk. In another aspect, the silk includes sericin, which is also a component of natural silk.

In one embodiment, the silk is secured only to the outside of the stent graft. In another embodiment, the silk is secured to distal regions of the stent graft.

The silk may be attached to the stent portion of the stent graft, or it may be attached to the graft portion of the stent graft, or it may be attached to both the stent and graft portions of the stent graft.

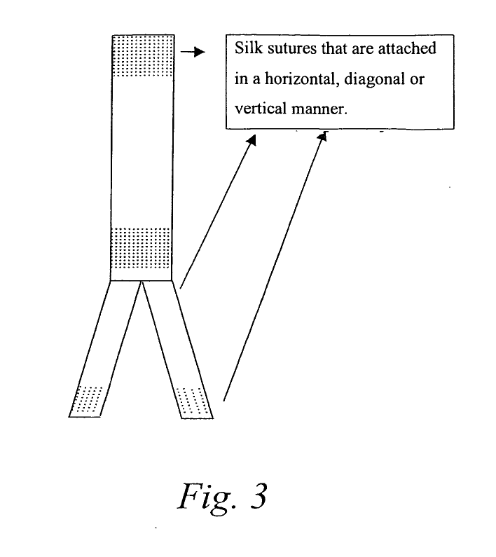

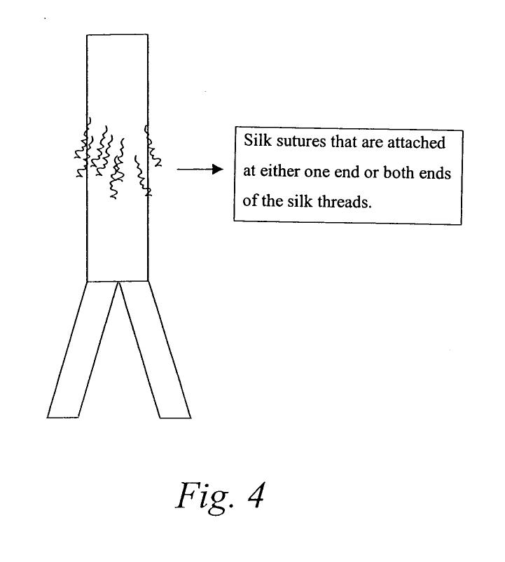

The silk threads can be located on the stent-graft in various configurations that may result in either partial or complete coverage of the exterior of the stent-graft. The threads could be attached around the ends of the stent-graft, as shown in FIG. 3. The silk threads can be attached in bands along the stent graft. The attachment could be in a vertical, horizontal or diagonal manner. Depending on the specific design of the stent graft, the polymeric thread(s) can be attached to either the stent component or the graft component of the stent graft device. Alternatively, or in addition, the silk thread may be allowed to extend some distance from the stent graft. For example, as shown in FIG. 4, only one end of the silk threads may be secured to the stent graft, thereby allowing the other end of the thread to extend away from the graft. Alternatively, both ends of the thread may be secured to a stent graft, however, the mid-portion of the thread is not secured to the stent graft, and the ends of the thread are secured at a sufficiently short distance from one another that the mid-portion is free to extend away from the stent graft.

In another embodiment, the ends of the silk threads can be attached to the stent graft, and/or one or more points along the silk thread can be attached to the stent graft. In yet another embodiment, the ends of the silk thread are not attached to the stent graft. Rather, one or more points along the silk thread are attached to the stent graft. In yet another embodiment, the silk thread(s) can be made into a preformed structure (e.g., mesh, looped bundle, and the like) that is then attached to the stent graft.

In one aspect, the invention provides a silk-containing stent graft in which the silk is present on the stent graft in an amount effective to induce a biological response in a host into which the stent graft has been inserted. The biological response may be manifested as a reduction in the risk of rupture of an aneurysm into which the stent graft has been placed. In another aspect, the biological response is manifested as a reduction in perigraft leakage. The enhanced effectiveness of a silk-containing stent graft may result from the silk inducing a cellular deposition between the stent graft and tissue adjacent to the stent graft. The cellular proliferation and/or extracellular matrix secretion progresses over time to form a cellular or non-cellular matrix, more commonly known as fibrotic tissue (i.e., tissue composed of fibroblasts, smooth muscle cells and extracellular matrix components such as collagen), which can hold the stent-graft in place within the vessel and/or act to fill part or all of the aneurysm.

The stent graft may, in addition to the silk, include a coating on some or all of the silk. The coating can degrade or dissolve over a period of time following insertion of the stent graft into a host. The presence of the coating functions to delay contact between the silk and the host. Suitable coatings for this purpose include, without limitation, gelatin, degradable polyesters (e.g., PLGA, PLA, MePEG-PLGA, PLGA-PEG-PLGA, copolymers and blends thereof), cellulose and cellulose derivatives (e.g., hydroxypropyl cellulose), polysaccharides (e.g., hyaluronic acid, dextran, dextran sulfate, chitosan), lipids, fatty acids, sugar esters, nucleic acid esters, polyanhydrides polyorthoesters and polyvinylalcohol (PVA). For example, in one embodiment of the invention, the silk is coated with a physical barrier. Such barriers can include biodegradable materials, such as gelatin, PLGA/MePEG film, PLA, polyethylene glycol, and the like. In the case of PLGA/ MePEG, once the PLGA/ MePEG becomes exposed to blood, the MePEG will dissolve out of the PLGA, leaving channels through the PLGA to the underlying layer of silk. The exposed silk layer then is available to initiate its biological activity.

In another embodiment, the stent graft can include a polymeric or non-polymeric coating that further comprises silk. The silk can be in the form of threads, short fibers, particles, or a combination thereof.

In another embodiment the stent graft can include polymeric fibers, yarns or threads that are attached to the stent graft. These fibers may be composed of polymers other than silk. Polymers that can be used include but are not limited to polyesters, such as DACRON, PTFE, nylon, poly(ethylene), poly(propylene) or degradable polyesters (e.g., PLGA, PCL, and poly(dioxanone)). These fibers can have one or more silk threads included in the polymeric fiber or yarn. In another embodiment, these threads, fibers or yarn can be coated with a polymeric or non-polymeric carrier that further contains silk fibers, threads or particles. The polymeric carriers can be degradable or non degradable. Examples of polymer carriers and non-polymeric carriers that can be used are described below.

In addition, or instead of containing a coating as described above, the silk-containing stent graft of the present invention may further include a biologically active agent that is capable of inducing a fibrotic response in a host into which the stent graft has been inserted. For example, the biologically active agent may induce an enhanced cellular deposition response and/or enhanced cellular matrix deposition. Exemplary agents include bleomycin and analogues and derivatives. Further representative examples include talcum powder, talc, ethanol, metallic beryllium and oxides thereof, copper, silk, silver nitrate, quartz dust, crystalline silicates and silica. Other agents which may be used include components of extracellular matrix, vitronectin, fibronectin, chondroitin sulphate, laminin, hyaluronic acid, elastin, fibrin, fibrinogen, bitronectin, proteins found in basement membrane, fibrosin, collagen, polylysine, vinyl chloride, polyvinyl chloride, poly(ethylene-co-vinylacetate), polyurethane, polyester (e.g., DACRON), and inflammatory cytokines such as TGFβ, PDGF, VEGF (including VEGF-2, VEGF-3, VEGF-A,VEGF-B and VEGFC), aFGF, bFGF, TNFα, NGF, GM-CSF, IGF-a, IL-1, IL-8, IL-6, growth hormone, EDGF (epidermal growth factor), and CTGF (connective tissue growth factor), and analogues and derivatives thereof, and adhesives, such as cyanoacrylate or a crosslinked poly(ethylene glycol)—methylated collagen composition, such as CT3 (Cohesion Technologies, Palo Alto, Calif.). Additional agents include naturally occurring or synthetic peptides containing the RGD (arginine-glycine-aspartic acid) residue sequence, and factors produced by immune cells such as Interleukin-2 (IL-2), Interleukin-4 (IL-4), Interleukin-1 (IL-1), Interleukin-8 (IL-8), Interleukin-6 (IL-6), Granulocyte-Monocyte Colony-Stimulating-Factor (GM-CSM), monocyte chemotactic protein, histamine and cell adhesion molecules including integrins, and bone morphogenic molecules including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15 and BMP-16. Of these BMP's, BMP-2, BMP-3, BMP-4, BMP-5, BMP-6 and BMP-7 are of particular utility. Other examples include peptide and non-peptide agonists of the above factors, and analogues and derivatives thereof, proteins, carbohydrates and peptides that contain cellular adhesion sequences, inorganic or organic small anionic molecule stimulants, and DNA or RNA sequences which promote the synthesis of proteins that stimulate cell growth.

In addition to, or instead of, containing a coating, as described above, the silk-containing stent graft of the present invention may further include a biologically active agent, wherein the agent induces an enhanced cellular proliferation response in a host into which the stent graft has been inserted. Representative examples of agents that stimulate cellular proliferation include, without limitation, dexamethasone, isotretinoin, 17-β-estradiol, diethylstibesterol, cyclosporin A and all-trans retinoic acid (ATRA) and analogues and derivatives thereof.

In another aspect of the invention, the biologically active agent may act to inhibit processes which result in breakdown of the tissue within the aneurysm which can delay or prevent expansion of the aneurysm. Examples of such therapeutic agents include, without limitation, caspase inhibitors (e.g., VX-799), MMP inhibitors (e.g., BATIMASTAT, also known as BB-94 and MARIMISTAT (both from British Biotech, UK) and TIMP's (tissue inhibitors of matrix metalloproteinases)), cytokine inhibitors (e.g., chlorpromazine, mycophenolic acid, rapamycin, 1α-hydroxy vitamin D₃), MCP-1 antagonists (e.g., nitronaproxen, Bindarit, 1-alpha-25 dihydroxy vitamin D₃), TNFa antagonists/TACE inhibitors (e.g., E-5531, AZD-4717, glycophosphopeptical, UR-12715, cilomilast, infliximab, lentinan, and etanercept ), IL-1, ICE and IRAK antagonists (e.g., E-5090, CH-172, CH-490, AMG-719, iguratimod, AV94-88, pralnacasan, esonarimod, and tranexamic acid), chemokine receptor antagonists (e.g., ONO-4128, L-381, CT-112, AS-900004, SCH-C, ZK-811752, PD-172084, UK-427857, SB-380732, vMIP II, SB-265610, DPC-168, TAK-779, TAK-220, and KRH-1120) and anti-inflammatory agents (e.g., dexamethasone, cortisone, fludrocortisone, prednisone, prednisolone, 6α-methylprednisolone, triamcinolone, and betamethasone) or analogues and derivatives thereof. It should be clear to one skilled in the art that these biologically active agents may be used individually or in combination or may be placed singly or in combination at various points within the stent-graft and that other agents which act as therapeutic agents to prevent expansion of the aneurysm can be applied.

Within further aspects of the present invention, the silk-containing stent grafts may include a polymeric carrier that is adapted to contain and release a therapeutic agent. Suitable polymeric carriers and therapeutic agents are described below.

In certain embodiments, the polymeric carrier may include regions, pockets, or granules that contain one or more hydrophobic compounds (e.g., therapeutic agents). For example, within one embodiment of the invention, hydrophobic compounds may be incorporated within a matrix, followed by incorporation of the matrix within the polymeric carrier. A variety of matrices can be utilized in this regard, including for example, carbohydrates and polysaccharides, such as starch, cellulose, dextran, methylcellulose, chitosan and hyaluronic acid, and proteins or polypeptides, such as albumin, collagen and gelatin. Within alternative embodiments, hydrophobic compounds may be contained within a hydrophobic core, and this core contained within a hydrophilic shell. These and other carriers and therapeutic agents are discussed in the next section.

As mentioned above, the stent graft may be of any type or configuration that is suitable for the medical purpose intended. In various exemplary aspects of the invention, the stent graft is bifurcated, the stent graft is a tube graft, the stent graft is cylindrical, the stent graft is self-expandable, and/or the stent graft is balloon-expandable.

In one aspect, the stent graft of the present invention is sterile. Many pharmaceuticals are manufactured to be sterile and this criterion is defined by the USP XXII <1211>. Sterilization in this embodiment may be accomplished by a number of means accepted in the industry and listed in the USP XXII <1211>, including gas sterilization or ionizing radiation. Sterilization may be maintained by what is termed aseptic processing, defined also in USP XXII <1211>. Acceptable gases used for gas sterilization include ethylene oxide. Acceptable radiation types used for ionizing radiation methods include gamma, for instance from a cobalt 60 source and electron beam. A typical dose of gamma radiation is 2.5 MRad.

### Methods for Making Silk Stent Grafts

Silk may be attached to a stent graft in any manner that creates a secure bond between the stent graft and the silk. This “bond” may be a chemical bond, but it may also be a mechanical bond, as described in further detail below. While the following description is in terms of threads, silk of other configuration may be applied by the same techniques.

The polymeric silk threads can be attached to the stent-graft in various configurations that may result in either partial or complete coverage of the exterior of the stent-graft. The threads could be attached around the ends of the stent-graft, as shown in FIG. 3. The attachment could be in a vertical, horizontal or diagonal manner. Depending on the specific design of the stent graft, the polymeric thread(s) can be attached to either the stent component or the graft component of the stent graft device.

In one embodiment, when the graft material is on the outer side of the stent, a preferred method of attachment is for the silk thread(s) to be attached to the graft material. In another embodiment, when the stent is exterior to the graft material, a preferred method of attachment is for the silk thread(s) to be attached to stent. The silk threads can be attached at a single point to the stent graft or they can be attached to the stent graft at multiple points. In addition, threads may be attached to the central portion of the stent graft which will ultimately be located in the aneurysm. It is also possible to use a combination of all the above-described attachment methods.

The threads can be attached to the graft and/or the stent material by use of any one or a combination of the following exemplary methods: use of an adhesive, thermal welding, stitching, wrapping, weaving, knotting and looping. In one aspect, an adhesive is used to secure the silk to the stent graft. In another aspect, thermal welding is used to secure the silk to the stent graft. In another aspect, stitching is used to secure the silk to the stent graft. In another aspect, wrapping is used to secure the silk to the stent graft. In another aspect, weaving is used to secure the silk to the stent graft. In another aspect, knotting is used to secure the silk to the stent graft. In another aspect, looping is used to secure the silk to the stent graft.

In another aspect, the silk can be woven or knitted into a sheet or tubular structure that is then attached to the exterior of the stent graft structure. This covering can cover the entire exterior portion of the stent graft or it can cover one or more specific portions of the stent graft. In one embodiment, the covering is fixed to the stent graft. The covering can be attached by knotting it or sewing it to the stent graft structure, by using an adhesive to fix it to the stent graft structure, or a combination of the above methods. In another embodiment, the covering is not fixed on the stent graft and is simply placed as an outer covering on the stent graft structure.

In one aspect, the stent graft may be coated with a silk-containing suspension, solution or emulsion. Examples of suitable emulsions or suspensions include aqueous formulations of commercially available silk powders (e.g., silk powder available from Silk Biochemical Co., Ltd. (China), Nantong Dongchang Chemical Industrial Co, Ltd. (China) and Wuxi Smiss Technology Co, Ltd. (China)), which have been formed into either a solution or an emulsion. Preferably, emulsions contain between about 5 to 50 wt. % solids.

In one embodiment, the silk threads can be coated with a material that delays the time it takes for the silk to come into contact with the surrounding tissue and blood. This will allow placement of the stent graft without concern of thrombotic events as a result of the silk threads. In one aspect, the coating material degrades or dissolves during the deployment of the stent, while in another aspect the coating material degrades or dissolves after the stent graft has been implanted. These coating materials can be either polymeric or non-polymeric. Examples of coating materials include, without limitation, gelatin, degradable polyesters (e.g., PLGA, PLA, MePEG-PLGA, PLGA-PEG-PLGA, copolymers and blends thereof), cellulose and cellulose derivatives (e.g., hydroxypropyl cellulose), polysaccharides (e.g., hyaluronic acid, dextran, dextran sulfate, chitosan), lipids, fatty acids, sugar esters, nucleic acid esters, polyanhydrides, polyorthoesters, and PVA.

The silk threads can be coated prior to attachment to the stent graft or they can be coated onto the silk threads once they have been attached to the stent graft. This can be accomplished by using a spray-coating or dip-coating process.

In another embodiment, silk particle can be incorporated into a polymeric or a non-polymeric carrier which is in turn coated onto the stent graft. The polymeric carriers can be either degradable or non-degradable. Examples of polymer carriers and non-polymeric carriers that can be used are described below.

In one embodiment, silk particles or silk fibers are added to a solution of the polymeric or non polymeric carrier. The carrier solution forms a suspension upon addition of the silk particles or silk fibers. This suspension can be applied to all or a portion of the stent graft by dipping, painting, or spraying.

In another embodiment the stent graft can include polymeric fibers, yarns or threads that are attached to the stent graft. These fibers may be composed of polymers other than silk, such as, e.g., DACRON, PTFE, nylon, poly(ethylene), poly(propylene) or degradable polyesters (e.g., PLGA, PCL, and poly(dioxanone)). These fibers can have one or more silk threads included in the polymeric fiber or yarn. In another embodiment, threads, fibers or yarn can be coated with a polymeric or non-polymeric carrier that further contains silk fibers, threads or particles. The polymeric carriers can be either degradable or non degradable. The polymeric or non-polymeric carrier can be dissolved in a solvent that will not substantially dissolve the polymeric fiber during the exposure of the polymeric fiber to the solvent. Pieces of silk fibers or threads and/or silk particles can be added to the carrier solution. If required, an emulsifying agent or a surfactant can be added to the solution to aid in the suspension of the fibers, threads or particles. The polymeric threads, fibers, or yarn can be coated with the silk-containing carrier composition by dipping the polymeric threads, fibers or yarns into the silk/carrier suspension or spraying the silk/carrier suspension onto the polymeric threads, fibers or yarns. These coated systems can then be air dried and if required can be vacuum dried. The coated polymeric threads, fibers or yarn then can be attached to the stent graft by methods disclosed herein.

In another embodiment, the polymeric thread, yarn, fiber, and/or the stent graft can be coated with a solution that contains a polymer or a non-polymeric carrier. The coating can be partially dried such that the coating is still soft and tacky. Silk thread, pieces of silk thread or silk powder then can be embedded into the soft coating. This can be accomplished by spraying the silk onto the soft coating, by rolling the coated form in the silk, by stamping the silk onto the coated form or by a combination of these processes. The silk coated form can be further dried to remove the residual solvent.

In one aspect of the invention, the graft (also referred to as a wrap or sheath) may be prepared entirely from silk, where in one aspect the silk is not a biological or genetically engineered spider silk. For example, the entire graft may be formed from a biological or genetically engineered silkworm silk. However, in a different aspect, the stent graft of the present invention contains a graft that is not made entirely of silk, however, silk is affixed to the stent graft. This is a preferred aspect because, e.g., the amount of silk affixed to the stent graft can be tailored to achieve the desired amount of biological response which is induced by the silk. Thus, in one aspect, the present invention provides a stent graft wherein the graft is not made entirely from silk (or is not made from silk at all), however silk is affixed to the stent graft in a manner as exemplified above. For example, the stent graft may contain a graft made from non-silk material, e.g., polyester, polyamide, hydrocarbon polymer (e.g., polyethylene and polypropylene), polyurethane or fluoropolymer (or other suitable material) and silk is affixed to either the stent or graft portion of the stent graft. In one aspect, the stent graft has a single graft, which in various separate embodiments may be woven within the stent, contained within the lumen of the stent, or be located exterior to the stent, where silk is affixed to this stent graft. In another aspect, the stent graft has two grafts, which in various embodiments may be woven within the stent, contained within the lumen of the stent, and/or be located exterior to the stent, where silk is affixed to this stent graft. When the stent graft contains two grafts, the silk is preferably affixed to the graft in a manner that will allow the silk to contact the vessel wall, e.g., it may be affixed to the sheath which is located exterior to the stent. As mentioned previously, in a preferred embodiment the silk is silkworm silk. For example, fibers of silkworm silk and fibers of a different material (polyester, polyamide, spider silk, etc.) may be combined together to form a sheath that is used to construct a stent graft of the present invention.

In one embodiment, the silk or the silk/carrier compositions may further contain a biologically active agent that reduces the probability of an immediate thrombotic event, where exemplary agents of this type include, without limitation, heparin and hydrophobic quaternary amine heparin (e.g., heparin-benzalkonium chloride, heparin-tridodecylmethylammonium chloride) complexes. The heparin or heparin complexes can be applied by dip coating or spray coating.

In another embodiment, the silk-containing thread, fiber, or yarn can further contain a biologically active agent that enhances a cellular response and/or a fibrotic response. The agents that can be used in the present invention are described below. These agents can be incorporated by dip coating or spray coating the silk-containing threads, fibers or yarn with a solution that contains the biologically active agent. This solution can be a true solution, a suspension, a dispersion or an emulsion. The biologically active agent(s) can also be incorporated into a secondary carrier. A solution, suspension, dispersion or emulsion or the biologically active agent/carrier can be applied by a dip coating or spray coating process. These agents can be applied to the entire external surface of the stent graft or to one or more specific locations on the stent graft.

In another embodiment, the biologically active agent or biologically active agent/secondary carrier (e.g., solution) can further comprise a polymer. This solution can be applied to the silk-containing thread, fiber or yarn.

In another embodiment, the biologically active agent and/or biologically active agent/secondary carrier can be incorporated into a polymeric or non-polymeric carrier solution that contains silk. The solvent for the carrier may or may not be a solvent for the added biologically active agent. In the case where the solvent is not a solvent for the biologically active agent, the biologically active agent will be in the form of a suspension. In the case where the solvent for the carrier is a solvent for the biologically active agent, a solution of the biologically active agent will be formed. In another embodiment, the solvent is a solvent for the biologically active agent, but the amount of the biologically active agent added to the solution is greater that the solubility limit of the biologically active agent. In this case, a saturated suspension of the biologically active agent will be formed. The silk- and biologically active agent-containing solution can be applied to the stent graft or the polymeric thread, fiber or yarn by a process of dip-coating or spray coating. The solution can be applied to all of the exterior of the stent graft or to one or more regions of the stent graft or polymeric thread, fiber or yarn.

In another embodiment, the coating includes a “biocompatible” polymer that is coated with a polymer or other biologically active agent that results in an enhanced cellular response.

In one embodiment, the silk-containing stent graft is coated with a composition or a compound which promotes fibrosis and/or restenosis.

In another embodiment, the silk-containing stent graft is coated with an agent that is not released from the stent graft but yet still results in an enhanced cellular and extracellular matrix deposition response. These agents can be coated directly onto the stent graft or they can be incorporated into a non-degradable polymeric carrier.

In one aspect, the silk-containing stent grafts of the present invention are coated with, or otherwise adapted to release an agent that induces adhesion to vessel walls. Stent grafts may be adapted to release such an agent by (a) directly affixing to the stent graft a desired agent or composition (e.g., by either spraying the stent graft with a polymer/agent film, or by dipping the stent graft into a polymer/agent solution, or by other covalent or noncovalent means); (b) by coating the stent graft with a substance such as a hydrogel which will in turn absorb the desired agent or composition; (c) by interweaving an agent- or composition-coated thread into the stent graft (e.g., a polymer which releases the agent formed into a thread); (d) by inserting a sleeve or mesh which is comprised of or coated with the desired agent or composition; (e) constructing the stent graft itself with the desired agent or composition; or (f) otherwise impregnating the stent graft with the desired agent or composition. Suitable fibrosis inducing agents may be readily determined based upon the animal models provided in Example 9 (Screening Protocol for Assessment of Perigraft Reaction), Example 14 (In vivo Evaluation of Perivascular PU Films Coated with Different Silk Suture Material), and Example 15 (.In vivo Evaluation of Perivascular Silk Powder).

Exemplary agents which can result in an enhanced cellular response and/or enhanced matrix deposition response, or more generally a scarring response, include bleomycin and analogues and derivatives. Further representative examples include talcum powder, talc, ethanol, metallic beryllium, copper, silk, silver nitrate, quartz dust, crystalline silicates and silica. Other agents which may be used include components of extracellular matrix, vitronectin, fibronectin, chondroitin sulphate, laminin, hyaluronic acid, elastin, fibrin, fibrinogen, bitronectin, proteins found in basement membrane, fibrosin, collagen, polylysine, vinyl chloride, polyvinyl chloride, poly(ethylene-co-vinylacetate), polyurethane, polyester (e.g., DACRON), and inflammatory cytokines such as TGFβ, PDGF, VEGF (including VEGF-2, VEGF-3, VEGF-A, VEGF-B and VEGFC), aFGF, bFGF, TNFα, NGF, GM-CSF, IGF-a, IL-1, IL-8, IL-6, growth hormone, EDGF (epidermal growth factor), and CTGF (connective tissue growth factor), and analogues and derivatives thereof and adhesives, such as cyanoacrylate or a crosslinked poly(ethylene glycol)—methylated collagen composition, such as CT3. Additional agents include naturally occurring or synthetic peptides containing the RGD (arginine-glycine-aspartic acid) residue sequence, and factors produced by immune cells such as Interleukin-2 (IL-2), Interleukin-4 (IL-4), Interleukin-1 (IL-1), Interleukin-8 (IL-8), Interleukin-6 (IL-6), Granulocyte-Monocyte Colony-Stimulating-Factor (GM-CSM), monocyte chemotactic protein, histamine and cell adhesion molecules including integrins, and bone morphogenic molecules including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15 and BMP-16. Of these BMP's, BMP-2, BMP-3, BMP-4, BMP-5, BMP-6 and BMP-7 are of particular utility. Furthermore, included are peptide and non-peptide agonists of the above factors, and analogues and derivatives thereof, proteins, carbohydrates or peptides that contain cellular adhesion sequences, cytokines, inorganic or organic small anionic molecule stimulants, and DNA or RNA sequences which promote the synthesis of proteins that stimulate cell growth.

In another embodiment, the silk-containing stent graft is coated with a composition or a compound which stimulates cellular proliferation on the exterior surface of the graft. Representative examples of agents that stimulate cellular proliferation include, without limitation, dexamethasone, isotretinoin, 17-β-estradiol, diethylstibesterol, cyclosporin A, all-trans retinoic acid (ATRA), and analogues and derivatives thereof.

In another embodiment, the silk-containing stent graft is coated with a composition or a compound which acts to inhibit processes which result in pathological change of the tissue within the aneurysm. The composition or compound thus can prevent expansion of the aneurysm. Agents which inhibit such processes, but not by way of limitation, include caspase inhibitors, MMP inhibitors, MCP-1 antagonists, TNFa antagonists/TACE inhibitors, apoptosis inhibitors, IL-1, ICE and IRAK antagonists, chemokine receptor antagonists and anti-inflammatory agents. The following are examples of such agents: Caspase inhibitors (e.g., VX-799); MMP inhibitors (e.g., D-9120, doxycycline (2-Naphthacenecarboxamide, 4-(dimethylamino)-1,4,4a,5,5a,6,11,12a-octahydro-3,5,10,12,12a-pentahydroxy-6-methyl-1,11-dioxo-[4S-(4Alpha,4aAlpha,5Alpha,5aAlpha,6Alpha,12aAlpha)]-[CAS]), BB-2827, BB-1101 (2S-allyl-N1-hydroxy-3R-isobutyl-N4-(1S-methylcarbamoyl-2-phenylethyl)-succinamide), BB-2983, solimastat (N′-[2,2-Dimethyl-1(S)-[N-(2-pyridyl)carbamoyl]propyl]-N4-hydroxy-2(R)-isobutyl-3(S)-methoxysuccinamide), BATIMASTAT (Butanediamide, N4-hydroxy-N1-[2-(methylamino)-2-oxo-1-(phenylmethyl)ethyl]-2-(2-methylpropyl)-3-[(2-thienylthio)methyl]-, [2R-[1(S*),2R*,3S*]]-[CAS]), CH-138, CH-5902, D-1927, D-5410, EF-13 (Gamma-linolenic acid lithium salt), CMT-3 (2-Naphthacenecarboxamide, 1,4,4a,5,5a,6,11,12a-octahydro-3,10,12,12a-tetrahydroxy-1,11-dioxo-, (4aS,5aR,12aS)-[CAS]), MARIMASTAT (N-[2,2-Dimethyl-1(S)-(N-methylcarbamoyl)propyl]-N,3(S)-dihydroxy-2(R)-isobutylsuccinamide), TIMP's (tissue inhibitors of matrix metalloproteinases), ONO-4817, rebimastat (L-Valinamide, N-((2S)-2-mercapto-1-oxo-4-(3,4,4-trimethyl-2,5-dioxo-1-imidazolidinyl)butyl)-L-leucyl-N,3-dimethyl-[CAS]), PS-508, CH-715, nimesulide (Methanesulfonamide, N-(4-nitro-2-phenoxyphenyl)-[CAS]), hexahydro-2-[2(R)-[1(RS)-(hydroxycarbamoyl)-4-phenylbutyl]nonanoyl]-N-(2,2,6,6-etramethyl-4-piperidinyl)-3(S)-pyridazine carboxamide, Rs-113-080, Ro-1130830, Cipemastat (1-Piperidinebutanamide, β-(cyclopentylmethyl)-N-hydroxy-Gamma-oxo-Alpha-[(3,4,4-trimethyl-2,5-dioxo-1-imidazolidinyl)methyl]-,(AlphaR,βR)-[CAS]), 5-(4′-biphenyl)-5-[N-(4-nitrophenyl)piperazinyl]barbituric acid, 6-methoxy-1,2,3,4-tetrahydro-norharman-1-carboxylic acid, Ro-31-4724 (L-Alanine, N-[2-[2-(hydroxyamino)-2-oxoethyl]-4-methyl-1-oxopentyl]-L-leucyl-, ethyl ester[CAS]), prinomastat (3-Thiomorpholinecarboxamide, N-hydroxy-2,2-dimethyl-4-((4-(4-pyridinyloxy)phenyl)sulfonyl)-, (3R)-[CAS]), AG-3433 (1H-Pyrrole-3-propanic acid, 1-(4′-cyano[1,1′-biphenyl]-4-yl)-b-[[[(3S)-tetrahydro-4,4-dimethyl-2-oxo-3-furanyl]amino]carbonyl]-, phenylmethyl ester, (bS)-[CAS]), PNU-142769 (2H-Isoindole-2-butanamide, 1,3-dihydro-N-hydroxy-Alpha-[(3S)-3-(2-methylpropyl)-2-oxo-1-(2-phenylethyl)-3-pyrrolidinyl]-1,3-dioxo-, (AlphaR)-[CAS]), (S)-1-[2-[[[(4,5-Dihydro-5-thioxo-1,3,4-thiadiazol-2-yl)amino]-carbonyl]amino]-1-oxo-3-(pentafluorophenyl)propyl]-4-(2-pyridinyl)piperazine, SU-5402 (1H-Pyrrole-3-propanoic acid, 2-[(1,2-dihydro-2-oxo-3H-indol-3-ylidene)methyl]-4-methyl-[CAS]), SC-77964, PNU-171829, CGS-27023A, N-hydroxy-2(R)-[(4-methoxybenzene-sulfonyl)(4-picolyl)amino]-2-(2-tetrahydrofuranyl)-acetamide, L-758354 ((1,1′-Biphenyl)-4-hexanoic acid, Alpha-butyl-Gamma-(((2,2-dimethyl-1-((methylamino)carbonyl)propyl)amino)carbonyl)-4′-fluoro-, (AlphaS-(AlphaR*,GammaS*(R*)))-[CAS]), GI-155704A, CPA-926 or an analogue or derivative thereof. Additional representative examples are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,691,381; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869; 6,087,359; Cytokine inhibitors (e.g., chlorpromazine, mycophenolic acid, rapamycin, 1α-hydroxy vitamin D₃); MCP-1 antagonists (e.g., nitronaproxen, Bindarit); TNFa antagonists/TACE inhibitors (e.g., E-5531 (2-Deoxy-6-0-[2-deoxy-3-0-[3(R)-[5(Z)-dodecenoyloxy]-decyl]-6-0-methyl-2-(3-oxotetradecanamido)-4-O-phosphono-β-D-glucopyranosyl]-3-0-[3(R)-hydroxydecyl]-2-(3-oxotetradecanamido)-Alpha-D-glucopyranose-1-O-phosphate), AZD-4717, glycophosphopeptical, UR-12715 (Benzoic acid, 2-hydroxy-5-[[4-[3-[4-(2-methyl-1H-imidazol[4,5-c]pyridin-1-yl]methyl]-1-piperidinyl]-3-oxo-1-phenyl-1-propenyl]phenyl]azo] (Z) [CAS]), PMS-601, AM-87, xyloadenosine (9H-Purin-6-amine, 9-B-D-xylofuranosyl-[CAS]), RDP-58, RDP-59, BB2275, benzydamine, E-3330 (Undecanoic acid, 2-[(4,5-dimethoxy-2-methyl-3,6-dioxo-1,4-cyclohexadien-1-yl)methylene]-, (E)-[CAS]), N-[D,L-2-(hydroxyaminocarbonyl) methyl-4-methylpentanoyl]-L-3-(2′-naphthyl)alanyl-L-alanine, 2-aminoethyl amide, CP-564959, MLN-608, SPC-839, ENMD-0997, Sch-23863 ((2-[10,11-Dihydro-5-ethoxy-5H-dibenzo [a,d] cyclohepten-S-yl]-N,N-dimethyl-ethanamine), SH-636, PKF-241-466, PKF-242-484, TNF-484A, cilomilastvb (Cis-4-cyano-4-[3-(cyclopentyloxy)-4-methoxyphenyl] cyclohexane-1-carboxylic acid), GW-3333, GW-4459, BMS-561392, AM-87, cloricromene (Acetic acid, [[8-chloro-3-[2-(diethylamino)ethyl]-4-methyl-2-oxo-2H-1-benzopyran-7-yl]oxy]-, ethyl ester [CAS]), thalidomide (1H-Isoindole-1,3(2H)-dione, 2-(2,6-dioxo-3-piperidinyl)-[CAS]), vesnarinone (Piperazine, 1-(3,4-dimethoxybenzoyl)-4-(1,2,3,4-tetrahydro-2-oxo-6-quinolinyl)-[CAS]), infliximab, lentinan, etanercept (1-235-Tumor necrosis factor receptor (human) fusion protein with 236-467-immunoglobulin G1 (human gammal-chain Fc fragment) [CAS]), diacerein (2-Anthracenecarboxylic acid, 4,5-bis(acetyloxy)-9,10-dihydro-9,10-dioxo-[CAS]) or an analogue or derivative thereof; IL-1, ICE & IRAK antagonists (e.g., E-5090 (2-Propenoic acid, 3-(5-ethyl-4-hydroxy-3-methoxy-1-naphthalenyl)-2-methyl-, (Z)-[CAS]), CH-164, CH-172, CH-490, AMG-719, iguratimod (N-[3-(Formylamino)-4-oxo-6-phenoxy-4H-chromen-7-yl] methanesulfonamide), AV94-88, pralnacasan (6H-Pyridazino(1,2-a)(1,2)diazepine-1-carboxamide, N-((2R,3S)-2-ethoxytetrahydro-5-oxo-3-furanyl)octahydro-9-((1-isoquinolinylcarbonyl)amino)-6,10-dioxo-, (1S,9S)-[CAS]), (2S-cis)-5-[Benzyloxycarbonylamino-1,2,4,5,6,7-hexahydro-4-(oxoazepino[3,2,1-hi]indole-2-carbonyl)-amino]-4-oxobutanoic acid, AVE-9488, ESONARIMOD (Benzenebutanoic acid, Alpha-[(acetylthio)methyl]-4-methyl-Gamma-oxo-[CAS], from Taisho Co., Japan), pralnacasan (6H-Pyridazino(1,2-a)(1,2)diazepine-1-carboxamide, N-((2R,3S)-2-ethoxytetrahydro-5-oxo-3-furanyl)octahydro-9-((1-isoquinolinylcarbonyl)amino)-6,10-dioxo-, (1S,9S)-[CAS]), tranexamic acid (Cyclohexanecarboxylic acid, 4-(aminomethyl)-, trans-[CAS]), Win-72052, Romazarit (Ro-31-3948) (Propanoic acid, 2-[[2-(4-chlorophenyl)-4-methyl-5-oxazolyl]methoxy]-2-methyl-[CAS]), PD-163594, SDZ-224-015 (L-Alaninamide N-((phenylmethoxy)carbonyl)-L-valyl-N-((1S)-3-((2,6-dichlorobenzoyl)oxy)-1-(2-ethoxy-2-oxoethyl)-2-oxopropyl)-[CAS]), L-709049 (L-Alaninamide, N-acetyl-L-tyrosyl-L-valyl-N-(2-carboxy-1-formylethyl)-, (S)-[CAS]), TA-383 (1H-Imidazole, 2-(4-chlorophenyl)-4,5-dihydro-4,5-diphenyl-, monohydrochloride, cis-[CAS]), EI-1507-1 (6a,12a-Epoxybenz[a]anthracen-1,12(2H,7H)-dione, 3,4-dihydro-3,7-dihydroxy-8-methoxy-3-methyl-[CAS]), Ethyl 4-(3,4-dimethoxyphenyl)-6,7-dimethoxy-2-(1,2,4-triazol-1-yl methyl)quinoline-3-carboxylate, EI-1941-1, TJ-114, anakinra (Interleukin 1 receptor antagonist (human isoform x reduced), N2-L-methionyl-[CAS]) ) or an analogue or derivative thereof; Chemokine receptor antagonists (e.g., ONO-4128 (1,4,9-Triazaspiro(5.5)undecane-2,5-dione, 1-butyl-3-(cyclohexylmethyl)-9-((2,3-dihydro-1,4-benzodioxin-6-yl)methyl-[CAS]), L-381, CT-112 (L-Arginine, L-threonyl-L-threonyl-L-seryl-L-glutaminyl-L-valyl-L-arginyl-L-prolyl-[CAS]), AS-900004, SCH-C, ZK-811752, PD-172084, UK-427857, SB-380732, vMIP II, SB-265610, DPC-168, TAK-779 (N, N-Dimethyl-N-[4-[2-(4-methylphenyl)-6,7-dihydro-5H-benzocyclohepten-8-ylcarboxamido]benyl]tetrahydro-2H-pyran-4-aminium chloride), TAK-220, KRH-1120) or an analogue or derivative, and anti-inflammatory agents (e.g., dexamethasone, cortisone, fludrocortisone, prednisone, prednisolone, 6α-methylprednisolone, triamcinolone, betamethasone), or analogues and derivatives thereof.

It should be clear to one skilled in the art that these biologically active agents may be used individually or in combination or may be placed singly or in combination at various points within the stent-graft and that other agents which act as a therapeutic agent to prevent expansion of the aneurysm can be applied.

Drugs and dosage: Therapeutic agents that may be used include but are not limited to: (A) Stimulators of cell proliferation (e.g., dexamethasone, isotretinoin, 17-β-estradiol, diethylstibesterol, cyclosporine A and all-trans retinoic acid (ATRA); (B) Caspase inhibitors (e.g. VX-799); (C) MMP Inhibitors (e.g., doxycycline, BATIMASTAT), (D) Cytokine inhibitors (e.g., chlorpromazine, mycophenolic acid, rapamycin, 1α-hydroxy vitamin D₃); (E) MCP-1 Antagonists (e.g., nitronaproxen, Bindarit); (F) TNFa Antagonists/TACE inhibitors (e.g., E-5531, AZD-4717, glycophosphopeptical, UR-12715, cilomilast, infliximab, lentinan, and etanercept); (G) IL1-ICE and IRAK antagonists (e.g., E-5090, CH-172, CH-490, AMG-719, iguratimod, AV94-88, pralnacasan, ESONARIMOD, tranexamic acid); (H) Chemokine receptor antagonists (e.g., ONO-4128, L-381, CT-112, AS-900004, SCH-C, ZK-811752, PD-172084, UK-427857, SB-380732, vMIP II, SB-265610, DPC-168, TAK-779, TAK-220, and KRH-1120); and (I) Anti-inflammatory agents (e.g., dexamethasone, cortisone, fludrocortisone, prednisone, prednisolone, 6α-methylprednisolone, triamcinolone, betamethasone).

Drugs are to be used at concentrations that range from several times more than to 10%, 5%, or even less than 1% of the concentration typically used in a single therapeutic systemic dose application. Preferably, the drug is released in effective concentrations for a period ranging from 1-90 days. (A) Stimulators of cell proliferation (e.g., dexamethasone, isotretinoin, 17-β-estradiol, diethylstibesterol, cyclosporin A, all-trans retinoic acid (ATRA) and analogues and derivatives thereof): total dose not to exceed 50 mg (range of 0.1 μg to 50 mg); preferred 1 μg to 10 mg. The dose per unit area of 0.01 μg -200 μg per mm²; preferred dose of 0.1 μg/mm²-20 μg/mm². Minimum concentration of 10⁻⁹-10⁻⁴ M of agent is to be maintained on the device surface. (B) Caspase inhibitors (e.g., VX-799 and analogues and derivatives thereof): total dose not to exceed 100 mg (range of 0.1 μg to 100 mg); preferred 1 μg to 25 mg. The dose per unit area of 0.01μg-500 μg per mm²; preferred dose of 0.1 μg/mm²-50 μg/mm². Minimum concentration of 10⁻⁹-10⁻⁴ M of agent is to be maintained on the device surface. (C) MMP Inhibitors (e.g., doxycycline, BATIMASTAT, and analogues and derivatives thereof): total dose not to exceed 100 mg (range of 0.1 μg to 100 mg); preferred 1 μg to 25 mg. The dose per unit area of 0.01 μg-500 μg per mm²; preferred dose of 0.1 μg/mm²-50 μg/mm². Minimum concentration of 10⁻⁹-10⁻⁴ M of agent is to be maintained on the device surface. (D) Cytokine inhibitors (e.g., chlorpromazine, mycophenolic acid, rapamycin, 1α-hydroxy vitamin D3, and analogues and derivatives thereof): total dose not to exceed 100 mg (range of 0.1 μg to 100 mg); preferred 1 μg to 25 mg. The dose per unit area of 0.01 μg-500 μg per mm²; preferred dose of 0.1 μg/mm²-50 μg/mm². Minimum concentration of 10⁻⁹-10⁻⁴ M of agent is to be maintained on the device surface. (E) MCP-1 Antagonists (e.g., nitronaproxen, Bindarit and analogues and derivatives thereof): total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of agent is to be maintained on the device surface. (F) TNFa Antagonists/TACE inhibitors (e.g., E-5531, AZD-4717, glycophosphopeptical, UR-12715, cilomilast, infliximab, lentinan, etanercept, and analogues and derivatives thereof): total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of agent is to be maintained on the device surface. (G) IL1-ICE and IRAK antagonists (e.g., E-5090, CH-172, CH-490, AMG-719, iguratimod, AV94-88, pralnacasan, ESONARIMOD, tranexamic acid, and analogues and derivatives thereof): total dose not to exceed 200 mg (range of 1.0 μg to 200 mg); preferred 1 μg to 50 mg. The dose per unit area of the device of 1.0 μg -100 μg per mm²; preferred dose of 2.5 μg/mm²-50 μg/mm². Minimum concentration of 10⁻⁸-10⁻⁴ M of agent is to be maintained on the device surface. (H) Chemokine receptor antagonists (e.g., ONO-4128, L-381, CT-112, AS-900004, SCH-C, ZK-811752, PD-172084, UK-427857, SB-380732, vMIP II, SB-265610, DPC-168, TAK-779, TAK-220, KRH-1120 or an analogue or derivative thereof): total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of agent is to be maintained on the device surface. (I) Anti-inflammatory agents (e.g., dexamethasone, cortisone, fludrocortisone, prednisone, prednisolone, 6α-methylprednisolone, triamcinolone, betamethasone, and analogues and derivatives thereof): total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of agent is to be maintained on the device surface.

Optionally, within one embodiment of the invention, the silk-containing stent graft of the invention may include a polymer, which may be either biodegradable or non-biodegradable. Representative examples of biodegradable compositions include albumin, collagen, gelatin, hyaluronic acid, starch, cellulose and cellulose derivatives (e.g., methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(ether ester) multiblock copolymers, based on poly(ethylene glycol) and poly(butylene terephthalate), tyrosine-derived polycarbonates (e.g., U.S. Pat. No. 6,120,491), poly(hydroxyl acids), poly(D,L-lactide), poly(D,L-lactide-co-glycolide), poly(glycolide), poly(hydroxybutyrate), polydioxanone, poly(alkylcarbonate) and poly(orthoesters), polyesters, poly(hydroxyvaleric acid), polydioxanone, poly(ethylene terephthalate), poly(malic acid), poly(tartronic acid), poly(acrylamides), polyanhydrides, polyphosphazenes, poly(amino acids), poly(alkylene oxide)-poly(ester) block copolymers (e.g., X-Y, X-Y-X or Y-X-Y, where X is a polyalkylene oxide and Y is a polyester (e.g., PLGA, PLA, PCL, polydioxanone and copolymers thereof) and their copolymers as well as blends thereof. [see generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986]. Representative examples of non-degradable polymers suitable for the delivery of fibrosing agents include poly(ethylene-co-vinyl acetate) (“EVA”) copolymers, silicone rubber, acrylic polymers [polyacrylic acid, polymethylacrylic acid, polymethylmethacrylate, poly(butyl methacrylate)], poly(alkylcynoacrylate) [e.g., poly(ethylcyanoacrylate), poly(butylcyanoacrylate) poly(hexylcyanoacrylate) poly(octylcyanoacrylate)], polyethylene, polypropylene, polyamides (nylon 6,6), polyurethane, poly(ester urethanes), poly(ether urethanes), poly(ester-urea), polyethers [poly(ethylene oxide), poly(propylene oxide), polyalkylene oxides (e.g., PLURONIC compounds from BASF Corporation, Mount Olive, N.J.), and poly(tetramethylene glycol)], styrene-based polymers [polystyrene, poly(styrene sulfonic acid), poly(styrene)-block-poly(isobutylene)-block-poly(styrene), poly(styrene)-poly(isoprene) block copolymers], and vinyl polymers (polyvinylpyrrolidone, poly(vinyl alcohol), poly(vinyl acetate phthalate) as well as copolymers and blends thereof. Polymers may be anionic (e.g., alginate, carrageenan, carboxymethyl cellulose, poly(acrylamido-2-methyl propane sulfonic acid) and copolymers thereof, poly(methacrylic acid and copolymers thereof and poly(acrylic acid) and copolymers and blends thereof), or cationic (e.g., chitosan, poly-L-lysine, polyethylenimine, and poly(allyl amine)) and copolymers and blends thereof (see generally, Dunn et al., J. Applied Polymer Sci. 50:353-365, 1993; Cascone et al., J. Materials Sci.: Materials in Medicine 5:770-774, 1994; Shiraishi et al., Biol. Pharm. Bull. 16(11):1164-1168, 1993; Thacharodi and Rao, Int'l J. Pharm. 120:115-118, 1995; Miyazaki et al., Int'l J. Pharm. 118:257-263, 1995). Particularly preferred polymeric carriers include poly(ethylene-co-vinyl acetate), polyurethanes, poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of poly (caprolactone) or poly (lactic acid) with a polyethylene glycol (e.g., MePEG), silicone rubbers, poly(styrene)block-poly(isobutylene)-block-poly(styrene), poly(acrylate) polymers and blends, admixtures, or co-polymers of any of the above. Other preferred polymers include collagen, poly(alkylene oxide)-based polymers, polysaccharides such as hyaluronic acid, chitosan and fucans, and copolymers of polysaccharides with degradable polymers.

Other representative polymers capable of sustained localized delivery of fibrosis-inducing agents include carboxylic polymers, polyacetates, polyacrylamides, polycarbonates, polyethers, polyesters, polyethylenes, polyvinylbutyrals, polysilanes, polyureas, polyurethanes, polyoxides, polystyrenes, polysulfides, polysulfones, polysulfonides, polyvinylhalides, pyrrolidones, rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxy, melamine, other amino resins, phenolic polymers, and copolymers thereof, water-insoluble cellulose ester polymers (including cellulose acetate propionate, cellulose acetate, cellulose acetate butyrate, cellulose nitrate, cellulose acetate phthalate, and mixtures thereof), polyvinylpyrrolidone, polyethylene glycols, polyethylene oxide, polyvinyl alcohol, polyethers, polysaccharides, hydrophilic polyurethane, polyhydroxyacrylate, dextran, xanthan, hydroxypropyl cellulose, methyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate compounds having hydrophilic esterifying groups, hydroxyacrylate, and acrylic acid, and combinations thereof. Other examples include cellulose esters and ethers, ethyl cellulose, hydroxyethyl cellulose, cellulose nitrate, cellulose acetate, cellulose acetate butyrate, cellulose acetate propionate, polyurethane, polyacrylate, natural and synthetic elastomers, rubber, acetal, nylon, polyester, styrene polybutadiene, acrylic resin, polyvinylidene chloride, polycarbonate, homopolymers and copolymers of vinyl compounds, polyvinylchloride, and polyvinylchloride acetate.

Representative examples of patents relating to drug-delivery polymers and their preparation include PCT Publication Nos. WO 98/19713, WO 01/17575, WO 01/41821, WO 01/41822, and WO 01/15526 (as well as their corresponding U.S. applications), U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,106,473, 6,110,483, 6,121,027, 6,156,345, 6,214,901, 6,368,611 6,630,155, 6,528,080, RE37,950, 6,46,1631, 6,143,314, 5,990,194, 5,792,469, 5,780,044, 5,759,563, 5,744,153, 5,739,176, 5,733,950, 5,681,873, 5,599,552, 5,340,849, 5,278,202, 5,278,201, 6,589,549, 6,287,588, 6,201,072, 6,117,949, 6,004,573, 5,702,717, 6,413,539, and 5,714,159, 5,612,052, and U.S. Publication Nos. 2003/0068377, 2002/0192286, 2002/0076441, and 2002/0090398.

It should be obvious to one of skill in the art that the polymers as described herein can also be blended or copolymerized in various compositions as required to deliver therapeutic doses of fibrosis-inhibiting agents.

Polymeric carriers for fibrosis-inhibiting agents can be fashioned in a variety of forms, with desired release characteristics and/or with specific properties depending upon the stent graft or composition being utilized. For example, polymeric carriers may be fashioned to release a fibrosing or other therapeutic agent upon exposure to a specific triggering event such as pH (see, e.g., Heller et al., “Chemically Self-Regulated Drug Delivery Systems,” in Polymers in Medicine III, Elsevier Science Publishers B. V., Amsterdam, 1988, pp. 175-188; Kang et al., J. Applied Polymer Sci. 48:343-354, 1993; Dong et al., J. Controlled Release 19:171-178, 1992; Dong and Hoffman, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers 1, Springer-Verlag, Berlin). Representative examples of pH-sensitive polymers include poly(acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and acrylmonomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water-soluble polymer.

Likewise, fibrosis-inducing and other therapeutic agents can be delivered via polymeric carriers which are temperature sensitive (see, e.g., Chen et al., “Novel Hydrogels of a Temperature-Sensitive PLURONIC Grafted to a Bioadhesive Polyacrylic Acid Backbone for Vaginal Drug Delivery,” in Proceed. Intern. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102, 1994; Okano et al., J. Controlled Release 36:125-133, 1995; Chun and Kim, J. Controlled Release 38:39-47, 1996; D'Emanuele and Dinarvand, Int'l J. Pharm. 118:237-242, 1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B. V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12):1997-2002, 1995).

Representative examples of thermogelling polymers, and their gelatin temperature [LCST (° C.)] include homopolymers such as poly(N-methyl-N-propylacrylamide), 19.8; poly(N-propylacrylamide), 21.5; poly(N-methyl-N-isopropylacrylamide), 22.3; poly(N-propylmethacrylamide), 28.0; poly(N-isopropylacrylamide), 30.9; poly(N,n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water-soluble polymers such as acrylmonomers (e.g., acrylic acid and derivatives thereof such as methylacrylic acid, acrylate and derivatives thereof such as butyl methacrylate, acrylamide, and N-butyl acrylamide).

Other representative examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, polyalkylene oxide-polyester block copolymers of the structure X-Y, Y-X-Y and X-Y-X, where X is a polyalkylene oxide and Y is a biodegradable polyester (e.g., PLG-PEG-PLG), and polyalkylene oxides, such as PLURONIC F-127, 10-15° C.; L-122, 19° C.; L-92, 26° C.; L-81, 20° C.; and L-61, 24° C. (BASF Corporation, Mount Olive, N.J.).

Representative examples of patents relating to thermally gelling polymers and their preparation include U.S. Pat. Nos. 6,451,346; 6,201,072; 6,117,949; 6,004,573; 5,702,717; and 5,484,610; and PCT Publication Nos. WO 99/07343; WO 99/18142; WO 03/17972; WO 01/82970; WO 00/18821; WO 97/15287; WO 01/41735; WO 00/00222 and WO 00/38651.

Fibrosis-inducing agents may be linked by occlusion in the matrices of the polymer, bound by covalent linkages, or encapsulated in microcapsules. Within certain embodiments of the invention, therapeutic compositions are provided in non-capsular formulations such as microspheres (ranging from nanometers to micrometers in size), pastes, threads of various size, films and sprays.

Within certain aspects of the present invention, the therapeutic composition is biocompatible and releases one or more fibrosis-inducing agents over a period of several hours, days, or, months. Further, therapeutic compositions of the present invention should preferably be stable for several months and capable of being produced and maintained under sterile conditions.

Within certain aspects of the present invention, therapeutic compositions may be fashioned in any size ranging from 50 nm to 500 μm, depending upon the particular use. These compositions can be in the form of microspheres, microparticles and/or nanoparticles. These compositions can be formed by spray-drying methods, milling methods, coacervation methods, W/O (water/oil) emulsion methods, W/O/W (water/oil/water) emulsion methods, and solvent evaporation methods. In another embodiment, these compositions can include microemulsions, emulsions, liposomes and micelles. Alternatively, such compositions may also be readily applied as a “spray”, which solidifies into a film or coating for use as a device surface coating or to line the tissues of the implantation site. Such sprays may be prepared from microspheres of a wide array of sizes, including for example, from 0.1 μm to 3 μm, from 10 μm to 30 μm, and from 30 μm to 100 μm.

Therapeutic compositions of the present invention may also be prepared in a variety of “paste” or gel forms. For example, within one embodiment of the invention, therapeutic compositions are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” may be readily made utilizing a variety of techniques (see, e.g., PCT Publication WO 98/24427). Other pastes may be applied as a liquid, which solidify in vivo due to dissolution of a water-soluble component of the paste and precipitation of encapsulated drug into the aqueous body environment. These “pastes” and “gels” containing fibrosis-inducing agents are particularly useful for application to the surface of tissues that will be in contact with the implant or device.

Within yet other aspects of the invention, the therapeutic compositions of the present invention may be formed as a film or tube. These films or tubes can be porous or non-porous. Preferably, such films or tubes are generally less than 5, 4, 3, 2, or 1 mm thick, more preferably less than 0.75 mm, 0.5 mm, 0.25 mm, or, 0.10 mm thick. Films or tubes can also be generated of thicknesses less than 50 μm, 25 μm or 10 μm. Such films are preferably flexible with a good tensile strength (e.g., greater than 50, preferably greater than 100, and more preferably greater than 150 or 200 N/cm²), good adhesive properties (i.e., adheres to moist or wet surfaces), and have controlled permeability. Fibrosis-inducing agents contained in polymeric films are particularly useful for application to the surface of a stent graft as well as to the surface of tissue, cavity or an organ.

Within certain embodiments of the invention, the therapeutic compositions may also include additional ingredients such as surfactants (e.g., PLURONICs F-127, L-122, L-101, L-92, L-81, and L-61), anti-inflammatory agents, antithrombotic agents, preservatives, antioxideants, and/ or anti-platelet agents.

Within certain embodiments, the composition may include radio-opaque or echogenic materials and magnetic resonance imaging (MRI) responsive materials (i.e., MRI contrast agents) to aid in visualization of the silk-containing stent graft under ultrasound, fluoroscopy and/or MRI. For example, a stent graft may be made with or coated with a composition which is echogenic or radiopaque (e.g., made with echogenic or radiopaque with materials such as powdered tantalum, tungsten, barium carbonate, bismuth oxide, barium sulfate, Metrazimide, Iopamidol, Iohexol, Iopromide, Iobitridol, Iomeprol, Iopentol, Ioversol, Ioxilan, Iodixanol, Iotrolan, Acetrizoic Acid derivatives, Diatrizoic Acid derivatives, lothalamic Acid derivatives, Ioxithalamic Acid derivatives, Metrizoic Acid derivatives, Iodamide, Iypophylic agents, Iodipamide and Ioglycamic Acid or, by the addition of microspheres or bubbles which present an acoustic interface). For visualization under MRI, contrast agents (e.g., Gadolinium (III) chelates or iron oxide compounds) may be incorporated into the stent graft, such as, for example, as a component in a coating or within the void volume of the device (e.g., within a lumen, reservoir, or within the structural material used to form the device).

Within further aspects of the present invention, polymeric carriers are provided which are adapted to contain and release a hydrophobic fibrosis-inducing compound, and/or the carrier containing the hydrophobic compound in combination with a carbohydrate, protein or polypeptide. Within certain embodiments, the polymeric carrier includes regions, pockets, or granules of one or more hydrophobic compounds. For example, within one embodiment of the invention, hydrophobic compounds may be incorporated within a matrix, followed by incorporation of the matrix within the polymeric carrier. A variety of matrices can be utilized in this regard, including for example, carbohydrates and polysaccharides such as starch, cellulose, dextran, methylcellulose, sodium alginate, heparin, chitosan and hyaluronic acid, proteins or polypeptides such as albumin, collagen and gelatin. Within alternative embodiments, hydrophobic compounds may be contained within a hydrophobic core, and this core contained within a hydrophilic shell.

Other carriers that may likewise be utilized to contain and deliver fibrosis-inducing agents described herein include: hydroxypropyl cyclodextrin (Cserhati and Hollo, Int. J. Pharm. 108:69-75, 1994), liposomes (see, e.g., Sharma et al., Cancer Res. 53:5877-5881, 1993; Sharma and Straubinger, Pharm. Res. 11(60):889-896, 1994; WO 93/18751; U.S. Pat. No. 5,242,073), liposome/gel (WO 94/26254), nanocapsules (Bartoli et al., J. Microencapsulation 7(2):191-197, 1990), micelles (Alkan-Onyuksel et al., Pharm. Res. 11(2):206-212, 1994), implants (Jampel et al., Invest. Ophthalm. Vis. Science 34(11):3076-3083, 1993; Walter et al., Cancer Res. 54:22017-2212, 1994, and U.S. Pat. No. 4,882,168), nanoparticles (Violante and Lanzafame PAACR), nanoparticles—modified (U.S. Pat. No. 5,145,684), nanoparticles (surface modified) (U.S. Pat. No. 5,399,363), micelle (surfactant) (U.S. Pat. No. 5,403,858), synthetic phospholipid compounds (U.S. Pat. No. 4,534,899), gas borne dispersion (U.S. Pat. No. 5,301,664), liquid emulsions, foam, spray, gel, lotion, cream, ointment, dispersed vesicles, particles or droplets, solid- or liquid-aerosols, microemulsions (U.S. Pat. No. 5,330,756), polymeric shell (nano- and micro-capsule) (U.S. Pat. No. 5,439,686), emulsions (Tarr et al., Pharm Res. 4: 62-165, 1987), and nanospheres (Hagan et al., Proc. Intern. Symp. Control Rel. Bioact. Mater. 22, 1995; Kwon et al., Pharm Res. 12(2):192-195; Kwon et al., Pharm Res. 10(7):970-974; Yokoyama et al., J. Contr. Rel. 32:269-277, 1994; Grefet al., Science 263:1600-1603, 1994; Bazile et al., J. Pharm. Sci. 84:493-498, 1994).

Within another aspect of the present invention, polymeric carriers may be materials that are formed in-situ. In one embodiment, the precursors can be monomers or macromers that contain unsaturated groups that can be polymerized. The monomers or macromers can then, for example, be injected into the treatment area or onto the surface of the treatment area and polymerized in-situ using a radiation source (e.g., visible light or UV light) or a free radical system (e.g., potassium persulfate and ascorbic acid or iron and hydrogen peroxide). The polymerization step can be performed immediately prior to, simultaneously with, or after injection of the reagents into the treatment site. Representative examples of compositions that undergo free radical polymerization reactions are described in PCT Publication Nos. WO 01/44307, WO 01/68720, WO 02/072166, WO 03/043552, WO 93/17669, and WO 00/64977, U.S. Pat. Nos. 5,900,245; 6,051,248; 6,083,524, 6,177,095; 6,201,065; 6,217,894; 6,166,130; 6,323,278; 6,639,014; 6,352,710; 6,410,645; 6,531,147; 5,567,435; 5,986,043; and 6,602,975, and U.S. Publication Nos. 2002/012796, 2002/0127266, 2002/0151650, 2003/0104032, 2002/0091229, and 2003/0059906.

In another embodiment, the reagents can undergo an electrophilic-nucleophilic reaction to produce a crosslinked matrix. For example, a 4-armed thiol derivatized polyethylene glycol can be reacted with a 4 armed NHS-derivatized polyethylene glycol under basic conditions (pH> about 8). Representative examples of compositions that undergo electrophilic-nucleophilic crosslinking reactions are described in U.S. Pat. Nos. 5,752,974; 5,807,581; 5,874,500; 5,936,035; 6,051,648; 6,165,489; 6,312,725; 6,458,889; 6,495,127; 6,534,591; 6,624,245; 6,566,406; 6,610,033; 6,632,457; U.S. Publication No. 2003/0077272; and co-pending patent applications entitiled “Tissue Reactive Compounds and Compositions and Uses Thereof (U.S. Serial No. 60/437,384, filed Dec. 30, 2002, and U.S. Serial No. 60/44,924, filed Jan. 17, 2003) and “Drug Delivery from Rapid Gelling Polymer Composition” (U.S. Serial No. 60/437,471, filed Dec. 30, 2002, and U.S. Serial No. 60/440,875, filed Jan. 17, 2003). Other examples of in-situ forming materials that can be used include those based on the crosslinking of proteins (described, e.g., in U.S. Pat. Nos. RE38158; 4,839,345; 5,514,379, 5,583,114; 6,458,147; 6,371,975, U.S. Publication Nos 2002/0161399 and 2001/0018598, and PCT Publication Nos. WO 03/090683; WO 01/45761; WO 99/66964, and WO 96/03159).

In another embodiment, the fibrosing agent can be coated onto all of the stent graft or a portion of the stent graft. This can be accomplished by dipping, spraying, painting or by vacuum deposition.

As described above, the fibrosing agent can be coated onto the stent graft using the polymeric coatings described above. In addition to the coating compositions and methods described above, there are various other coating compositions and methods that are known in the art. Representative examples of these coating compositions and methods are described in U.S. Pat. Nos. 6,610,016; 6,358,557; 6,306,176; 6,110,483; 6,106,473; 5,997,517; 5,800,412; 5,525,348; 5,331,027; 5,001,009; 6,562,136; 6,406,754; 6,344,035; 6,254,921; 6,214,901; 6,077,698; 6,603,040; 6,278,018; 6,238,799; 6,096,726; 5,766,158; 5,599,576; 4,119,094; 4,100,309; 6,599,558; 6,369,168; 6,521,283; 6,497,916; 6,251,964; 6,225,431; 6,087,462; 6,083,257; 5,739,237; 5,739,236; 5,705,583; 5,648,442; 5,645,883; 5,556,710; 5,496,581; 4,689,386; 6,214,115; 6,090,901; 6,599,448; 6,054,504; 4,987,182; 4,847,324; and 4,642,267; U.S. Publication Nos. 2003/0129130, 2001/0026834; 2003/0190420; 2001/0000785; 2003/0059631; 2003/0190405; 2002/0146581; 2003/020399; 2003/0129130, 2001/0026834; 2003/0190420; 2001/0000785; 2003/0059631; 2003/0190405; 2002/0146581; 2003/020399, and PCT Publication Nos. WO 02/055121; WO 01/57048; WO 01/52915;and WO 01/01957.

Within another aspect of the invention, the biologically active agent can be delivered with non-polymeric agents. These non-polymeric agents can include sucrose derivatives (e.g., sucrose acetate isobutyrate, sucrose oleate); sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols, such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols, calcium phosphate, sintered and unscintered hydoxyapatite, zeolites, paraffin wax; and combinations and mixtures thereof.

Representative examples of patents relating to non-polymeric delivery systems and their preparation include U.S. Pat. Nos. 5,736,152; 5,888,533; 6,120,789; 5,968,542; and 5,747,058.

The fibrosis-inducing agent may be delivered as a solution and may be incorporated directly into the solution to provide a homogeneous solution or dispersion. In certain embodiments, the solution is an aqueous solution. The aqueous solution may further include buffer salts, as well as viscosity modifying agents (e.g., hyaluronic acid, alginates, carboxymethyl cellulose (CMC), and the like). In another aspect of the invention, the solution can include a biocompatible solvent, such as ethanol, DMSO, glycerol, PEG-200, PEG-300 or NMP.

Within another aspect of the invention, the fibrosis-inhibiting agent can further include a secondary carrier. The secondary carrier can be in the form of microspheres (e.g., PLGA, PLLA, PDLLA, PCL, gelatin, polydioxanone, poly(alkylcyanoacrylate)), nanospheres (PLGA, PLLA, PDLLA, PCL, gelatin, polydioxanone, poly(alkylcyanoacrylate)), liposomes, emulsions, microemulsions, micelles (SDS, block copolymers of the form X-Y, X-Y-X or Y-X-Y where X is a poly(alkylene oxide) or alkyl ether thereof and Y is a polyester (e.g., PLGA, PLLA, PDLLA, PCL, and polydioxanone), zeolites or cyclodextrins.

The composition may further include preservatives, stabilizers, and dyes. In one aspect, the compositions of the present invention include one or more preservatives or bacteriostatic agents present in an effective amount to preserve a composition and/or inhibit bacterial growth in a composition, for example, bismuth tribromophenate, methyl hydroxybenzoate, bacitracin, ethyl hydroxybenzoate, propyl hydroxybenzoate, erythromycin, chlorocresol, benzalkonium chlorides, and the like. Examples of preservatives include paraoxybenzoic acid esters, chlorobutanol, benzylalcohol, phenethyl alcohol, dehydroacetic acid, sorbic acid, and the like. In one aspect, the compositions of the present invention include one or more bactericidal (also known as bacteriacidal) agents.

A variety of excipients may be added to impart specific properties to the formulation including, e.g., colorants, antioxidants, preservatives, binders to form granules, pore formers, density, tonicity, pH or osmotic pressure adjusting materials, or degradation accelerants such as acids or bases. In certain embodiments, the compositions of the invention may further include water and/or have have a pH of about 3-9.

### Methods for Utilizing Stent Grafts

Silk stent grafts of the present invention may be utilized to induce a perigraft reaction or to otherwise create a tight adhesive bond between an endovascular prosthesis and the vascular wall in a host. Such grafts are capable of providing a solution to the following common problems associated with endovascular stent graft technology.

1. Persistent Perigraft Leaks—a formation of fibrotic response or adhesion or tight adhesive bond between the proximal and distal interfaces between the stent portion of the stent graft and the vessel wall results in a more efficacious sealing around the device, and prevents late perigraft leaks arising at either end of the device even with a change in aneurysm morphology. Moreover, formation of a fibrous response or tight adhesion between the body of the graft and the aneurysm itself may result in occlusion of, or prevention of a perigraft leak due to retrograde flow (i.e., persistence of, or late reopening of the inferior mesenteric artery or lumbar arteries extending into the aneurysm).

2. Size of the Delivery Device—one difficulty with present delivery devices is that they are quite large due to the required thickness of the stent graft. By inducing a reaction in the wall, which in itself conveys strength to the graft portion of the stent graft prosthesis, a thinner graft material may be utilized in stent grafts of the present invention compared to standard stent grafts. Thus, in the various aspects of the invention, the silk stent graft has a thickness of less than 24 French, or less than 23 French, or less than 22 French, or less than 21 French, or less than 20 French.

3. Anatomic Factors which limit Patients with Aneurismal Disease who are Candidates for Treatment with Endovascular Stent Grafts—by inducing a fibrotic reaction or creating a tight durable adhesive bond between the prosthesis and the vascular wall at the proximal and distal margins of the grafted portion of the prosthesis, the length of the neck, particularly the proximal neck, can be shorter than the presently suggested 1.5 centimeters. This benefit is realized because the fibrotic reaction or tight adhesion between graft and vessel wall will enhance sealing of the graft even when there is a short length of contact between the graft and vessel wall. In an aneurysm, the walls are dilated and thus extend away from the graft. When there is a long neck, apposition between graft material and vessel wall is only between the portion of vessel wall of “normal” diameter. In some cases, the portion of the vessel to which the device is to be anchored is dilated, e.g., a dilated iliac artery distal to an abdominal aortic aneurysm. If this segment of the vessel is too dilated, it tends to continue expansion after graft insertion, resulting in late perigraft leaks. Patients with dilated iliac arteries or aortic neck might be denied therapy with uncoated devices but can advantageously receive a silk-containing stent graft of the present invention. Creation of a firm bond between the graft and the vessel wall will prevent the neck from expanding further.

4. Stent Graft Migration—as the silk stent graft of the present invention becomes firmly fixed against the vessel wall by more than just hooks or force of expansion between the stent graft and the vessel wall, migration of the stent graft or portions of the stent graft is prevented or reduced.

5. Expansion of Applications of Stent Grafts—Present applications of stent grafts for practical purposes are limited to situations where the stent graft is wholly deployed within a blood vessel. By strengthening the seal between the blood vessel wall and the device, this expands the possibility that the device can be used as an extravascular or even extra-anatomic conduit such as, but not limited to, between arteries, between an artery and a vein, or between veins, or between a vein and the peritoneal cavity. The expansion of stent grafts for these purposes is limited at least partially by the risk of leak of bodily fluid such as blood because of poor sealing at the site where the stent graft enters of leaves a body tube such as a blood vessel) or cavity.

Thus, stent grafts, which are adapted by the inclusion of silk to adhere to vessel walls, can be utilized in a wide variety of therapeutic applications. For example, a silk stent graft can be utilized to connect one artery to another, either intra-anatomically, e.g., to bypass aneurysms (e.g., carotid artery, thoracic aorta, abdominal aorta, subclavian artery, iliac artery, coronary artery, venous); to treat dissections (e.g., carotid artery, coronary artery, iliac artery, subclavian artery); to bypass long segment disease (e.g., carotid artery, coronary artery, aorta, iliac artery, femoral artery, popliteal artery), or to treat local rupture (e.g., carotid artery, aorta, iliac artery, renal artery, femoral artery). Silk stent grafts may also be utilized extra-anatomically, for example, for arterial-to-arterial dialysis fistula; or for percutaneous bypass grafts.

Stent grafts of the present invention may also be utilized to connect an artery to a vein (e.g., a dialysis fistula), or one vein to another (e.g., a portacaval shunt or venous bypass).

### A. Abdominal Aortic Aneurysms

In one representative example, silk stent grafts may be inserted into an Abdominal Aorta Aneurysm (AAA), in order to treat or prevent rupture of the abdominal aorta. Briefly, using sterile conditions, under appropriate anesthesia and analgesia, the common femoral artery is surgically exposed and an arteriotomy is performed after clamping of the artery. A guide wire is manipulated through the iliac arterial system and over this a catheter is inserted into the proximal abdominal aorta and an angiogram or intravascular ultrasound is performed. Subsequently the diagnostic catheter is exchanged over a guide wire for a delivery system, usually a sheath, containing the aortic portion of the stent graft system. If the device is an articulated bifurcated system, the most common iteration, than the ipsilateral iliac portion of the prosthesis is connected to the aortic portion. The device is deployed by releasing it from its constrained configuration, in the case of a stent graft composed of self-expanding stents. If the stent graft skeleton is composed of balloon expandable stents, it is released by withdrawal of the sheath and inflating a balloon to expand the stent graft in place. After release of the aortic and ipsilateral iliac portion of the prosthesis, surgical exposure and cut down of the opposite iliac artery is performed and a guide wire is manipulated so that it passes through the deployed portion of the prosthesis. A similar delivery device containing the contralateral iliac limb of the prosthesis is then manipulated into the deployed aortic portion of the prosthesis and under fluoroscopic guidance is released in an appropriate position. The position is chosen so that the entire grafted portion of the stent graft sits below the renal arteries and preferably is deployed above the internal iliac arteries although one or both may be occluded. Depending on the patient's anatomy, further limb extensions may be inserted on either side. If the device is a tube graft, or a one piece bifurcated device, insertion via only one femoral artery may be required. A final angiogram is normally obtained by an angiographic catheter position with its distal portion in the upper abdominal aorta.

### B. Thoracic Aortic Aneurysm or Dissection

In another representative example, a stent graft may be utilized to treat or prevent a thoracic aortic aneurysm. Briefly, under appropriate anesthesia and analgesia, using sterile technique, a catheter is inserted via the right brachial artery into the ascending thoracic aorta and an angiogram performed. Once the proximal and distal boundaries of the diseased segment of the aorta to be treated are defined, an operative exposure of one of the common femoral arteries, usually the right, and an operative arteriotomy is performed. A guide wire is manipulated through the diseased segment of the aorta and over this, the delivery device, usually a sheath, is advanced so that the device is positioned across the diseased segment with the grafted portion of the stent immediately below the origin of the left subdlavian artery. After contrast is injected to define the precise position of the stent graft, the device is deployed usually by withdrawing an outer sheath in the case of self-expanding stents so that the device is positioned immediately distal to the left subclavian artery and with its distal portion extending beyond the diseased portion of the thoracic aorta but above the celiac axis. A final angiogram is performed via the catheter inserted by the right brachial artery. The vascular access wounds are then closed.

### C. Delay of Onset of Activity of the Stent Coating

The time it takes to insert the device can be very long. For instance, it theoretically could be hours between the time that the first part of a device (usually the aortic segment) is deployed and the second part of the device is deployed. It is not until all the parts of the device are inserted that an adequate exclusion of the aneurysm is achieved. In other words, the coating on the device may cause blood clots to form on or around the device. Because blood is rushing around as well as through the device until it is fully deployed, thereby excluding the aneurysm, such blood clots could be dislodged and washed downstream, or, might propagate distally. This could result in the inadvertent and undesirable occlusion or partial occlusion of blood vessels downstream from the intended site of insertion of the device, which the operator had intended to keep open. Several strategies may be employed to address such difficulties.

For example, as discussed in more detail above, stent grafts may be constructed which are designed to delay the onset of activity of the fibrosis inducing, and/or fibrosis forming response to the silk (e.g., by coating the stent graft with a material such as heparin or PLGA which delays adhesion or fibrosis).

The following examples are offered by way of illustration, and not by way of limitation.

### EXAMPLES

### Example 1

### Attachment of Silk Braid to a Stent Graft—Hot Melt Glue

Silk braid (Ethicon Inc., 4-0, 638) was cut into lengths of approx 10 cm lengths. The end of a length of the silk braid was secured to the graft material of a stent graft (WALLGRAFT Endoprosthesis, Ref: 50019, Boston Scientific, Natick, Mass.) using a hot melt glue. The stent graft was then elongated and the silk braid was secured to the graft portion of the stent graft at approx. 2 cm spacings using the hot melt glue. The excess silk at the end was removed using a pair of scissors. The attachment of the silk was continued until 8 strands of silk were attached to the stent graft. Upon release of the stent graft from the elongated conformation, the contraction of the stent graft resulted in the silk braid forming protruding loops from the surface of the graft.

### Example 2

### Attachment of Silk Braid to a Stent Graft—Sutures

Silk braid (Ethicon Inc., 4-0, 638) was cut into approx 10 cm lengths. The end of a length of the silk braid was secured to the graft material of a stent graft (WALLGRAFT Endoprosthesis, Ref: 50019, Boston Scientific) using a PROLENE 7-0 suture (Ethicon Inc.). The silk braid was secured to the graft portion of the stent graft at approx. 2 cm spacings using additional PROLENE 7-0 sutures in such a manner that the silk braid formed loops that protruded from the stent graft's exterior surface. The excess silk at the end was removed using a pair of scissors. The attachment of the silk was continued until 8 strands of silk were attached to the stent graft.

### Example 3

### Coating of the Silk Braid with a Biologically Agent—Direct Dipping

Silk braid (Ethicon Inc., 4-0, 638) was cut into approx 10 cm lengths. The silk braid was dipped into a methanol solution of bleomycin. The concentration of the bleomycin in the methanol solution was altered from 0.1% to a saturated solution. The silk braid was immersed in the bleomycin solution for 5 minutes. The silk braid was then removed and air-dried. The bleomycin-loaded silk braid was then further dried under vacuum. The silk braid was then attached to the graft portion of the stent graft using PROLENE 7-0 sutures as described in Example 2.

### Example 4

### Coating of the Silk Braid with a Polymer/Biologically Agent—Direct Dipping

Silk braid (Ethicon Inc., 4-0, 638) is cut into approx 10 cm lengths. The silk braid is dipped into an ethyl acetate solution of poly(lactide-co-glycolide) [PLGA] (9K, 50:50, Birmingham Polymers) and bleomycin. The concentration of the PLGA is altered from 0.1% to 20% (w/v) and concentration of the bleomycin in the solution is altered from 0.1% to a saturated solution. The silk braid is immersed in the PLGA/bleomycin solution for 5 minutes. The silk braid is then removed and air-dried. The bleomycin loaded silk braid is then further dried under vacuum. The silk braid is then attached to the graft portion of the stent graft using PROLENE 7-0 sutures as described in Example 2.

### Example 5

### Coating of the Stent Graft with a Biologically Active Agent and Attachment of Polymeric Threads

A stent graft (WALLGRAFT Endoprosthesis, Ref: 50019, Boston Scientific) is pushed onto a 1 mL plastic pipette tip. The open end of the pipette tip is attached to a stainless steel rod that is attached to a Fisher overhead stirrer that is orientated horizontally. The stirrer is set to rotate at 30 rpm. A 2% PLGA (9K, 50:50, Birmingham Polymers) solution (ethyl acetate) that contains bleomycin is sprayed onto the rotating stent graft using an airbrush spray device. The concentration of the bleomycin in the PLGA solution is altered from 0.1% to a saturated solution. After the spraying process, the stent graft is allowed to air dry for 30 minutes while still rotating. The stent graft is then removed from the pipette tip and is further dried under vacuum for 24 h. Silk braid is then attached to the coated stent graft as described in Example 2.

### Example 6

### Preparation of Silk Powder

Several pieces of silk braid (Ethicon, 4-0, 638) are cut into lengths of approx 0.4 cm. These cut pieces are placed in a 100 mL round bottom flask that contains 50 mL 2M NaOH. The sample is stirred using a magnetic stirrer at room temperature for 24 h. The sample is neutralized using concentrated HCl. The neutralized contents are then dialyzed against deionized water using cellulose-based dialysis tubing (WMCO approx 3000) (NBS Biologicals-Spectrum Laboratories). The sample is dialyzed for 48 hours with 5 water changes. The dialyzed sample is then poured into a 100 mL round bottom flask. The sample is frozen and freeze-dried to yield a fluffy powdered material.

### Example 7

### Coating of the Stent Graft with a Powdered Silk/PLGA Coating

A stent graft (WALLGRAFT Endoprosthesis, Ref: 50019, Boston Scientific) is pushed onto a 1 mL plastic pipette tip. The open end of the pipette tip is attached to a stainless steel rod that is attached to a Fisher overhead stirrer that is orientated horizontally. The stirrer is set to rotate at 30 rpm. A 2% PLGA (9K, 50:50, Birmingham Polymers, Birmingham, Ala.) solution (ethyl acetate) that contains the powdered silk is sprayed onto the rotating stent graft using an airbrush spray device. The concentration of the powdered silk in the PLGA solution is altered from 0.1% to 50%. After the spraying process, the stent graft is allowed to air dry for 30 minutes while still rotating. The stent graft is then removed from the pipette tip and is further dried under vacuum for 24 h.

### Example 8

### Coating a Polymeric Thread with a Silk Powder/Carrier

A 2.5% (w/v) ChonoFlex AL 85A (CardioTech International Inc., Woburn, Mass.) solution in THF was prepared. Various amounts of silk powder (5-60% w/w compared to the ChronoFlex) were added to the polymer solution. A nylon suture (4-0 Black Monofilament Nylon (Ethicon Inc.) was pulled through the polymer silk solution. The coated suture was allowed to air-dry, after which it was further dried under vacuum. The coated suture was then attached to the graft portion of the stent graft using Prolene 7-0 sutures as described in Example 2.

### Example 9

### Screening Procedure for Assessment of Perigraft Reaction

Large domestic rabbits are placed under general anesthetic. Using aseptic precautions, the infrarenal abdominal aorta is exposed and clamped at its superior and inferior aspects. A longitudinal arterial wall arteriotomy is performed and a 2 millimeter diameter, 1 centimeter long segment of PTFE graft is inserted within the aorta and the proximal and distal aspect of the graft is sewn so that the entire aortic blood flow is through the graft which is contained in the abdominal aorta in the manner of open surgical abdominal aortic repair in humans (except that no aneurysm is present in this model). The aortotomy is then surgically closed and the abdominal wound closed and the animal recovered.

The animals are randomized to receive standard PTFE grafts, silk stent grafts, or silk stent grafts coated with other agents as described above.

The animals are sacrificed between 1 and 6 weeks post surgery, the aorta is removed en bloc and the area in relation to the graft is grossly examined for adhesive reaction. Any difference in morphology or histology of the vessel wall from portions of the artery that contain no graft, portion which contain graft without coating, and portion which contained graft with coating is noted.

### Example 10

### Screening Assay for Assessing the Effect of Cyclosporin a on Cell Proliferation

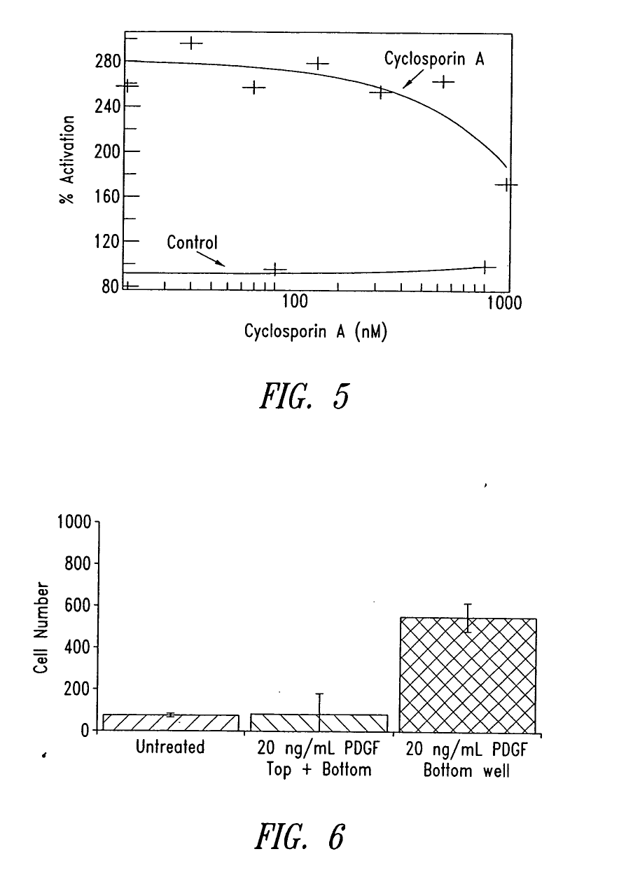

Smooth muscle cells at 70-90% confluency are trypsinized, replated at 600 cells/well in media in 96-well plates and allowed to attachment overnight. Cyclosporin A is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing smooth muscle cells and cyclosporin A are incubated at 37° C. for 72 hours. To terminate the assay, the media is removed by gentle aspiration. A 1/400 dilution of CYQUANT 400X GR dye indicator (Molecular Probes; Eugene, Oreg.) is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜480 nm excitation wavelength and ˜520 nm emission maxima. Activation of proliferation is determined by taking the average of triplicate wells and comparing average relative fluorescence units to the DMSO control. The results of the assay are shown in FIG. 5. References: In vitro toxicol. (1990) 3: 219; Biotech. Histochem. (1993) 68: 29; Anal. Biochem. (1993) 213: 426.

### Example 11

### Screening Assay for Assessing the Effect of PDGF on Smooth Muscle Cell Migration

Primary human smooth muscle cells are starved of serum in smooth muscle cell basal media containing insulin and human basic fibroblast growth factor (bFGF) for 16 hours prior to the assay. For the migration assay, cells are trypsinized to remove cells from flasks, washed with migration media and diluted to a concentration of 2-2.5×10⁵ cells/mL in migration media. Migration media consists of phenol red free Dulbecco's Modified Eagle Medium (DMEM) containing 0.35% human serum albumin. A 100 μL volume of smooth muscle cells (approximately 20,000-25,000 cells) is added to the top of a Boyden chamber assembly (QCM Chemotaxis 96-well migration plate; Chemicon International Inc., Temecula, Calif.). To the bottom wells, the chemotactic agent, recombinant human platelet derived growth factor (rhPDGF-BB) is added at a concentration of 10 ng/mL in a total volume of 150 μL. Paclitaxel is prepared in DMSO at a concentration of 10⁻² M and serially diluted 10-fold to give a range of stock concentrations (10⁻⁸ M to 10⁻² M). Paclitaxel is added to cells by directly adding paclitaxel DMSO stock solutions, prepared earlier, at a 1/1000 dilution, to the cells in the top chamber. Plates are incubated for 4 hours to allow cell migration.

At the end of the 4 hour period, cells in the top chamber are discarded and the smooth muscle cells attached to the underside of the filter are detached for 30 minutes at 37° C. in Cell Detachment Solution (Chemicon). Dislodged cells are lysed in lysis buffer containing the DNA binding CYQUANT GR dye and incubated at room temperature for 15 minutes. Fluorescence is read in a fluorescence microplate reader at ˜480 nm excitation wavelength and ˜520 nm emission maxima. Relative fluorescence units from triplicate wells are averaged after subtracting background fluorescence (control chamber without chemoattractant) and average number of cells migrating is obtained from a standard curve of smooth muscle cells serially diluted from 25,000 cells/well down to 98 cells/well. Inhibitory concentration of 50% (IC₅₀) is determined by comparing the average number of cells migrating in the presence of paclitaxel to the positive control (smooth muscle cell chemotaxis in reponse to rhPDGF-BB). The results of the assay are shown in FIG. 6. References: Biotechniques (2000) 29: 81; J. Immunol Methods (2001) 254: 85

### Example 12

### Animal Abdominal Aortic Aneurysm Model

Pigs or sheep are placed under general anesthetic. Using aseptic precautions the abdominal aorta is exposed. The animal is heparinized and the aorta is cross-clamped below the renal arteries and above the bifurcation. Collaterals are temporarily controlled with vessel loops or clips that are removed upon completion of the procedure. A longitudinal aortotomy is created in the arterial aspect of the aorta, and an elliptical shaped patch of rectus sheath from the same animal is sutured into the aortotomy to create an aneurysm. The aortic clamps from the lumbar arteries and collaterals are removed and the abdomen closed. After 30 days, the animal is reanesthesized and the abdominal wall again opened. A cutdown is performed on the iliac artery and through this, a stent graft is positioned across the infrarenal abdominal aorta aneurysm extending from normal infrarenal abdominal aorta above to normal infrarenal abdominal aorta below the surgically created aneurysm and the device is released in a conventional way.

Animals are randomized into groups of 5 receiving uncoated stent grafts, and 5 animals that receive a silk-containing stent graft. After closure of the arteriotomy and of the abdominal wound, the animal is allowed to recover. At 6 weeks and 3 months post stent graft insertion, the animal is sacrificed and the aorta removed en bloc. The infrarenal abdominal aorta is examined for evidence of histological reaction and perigraft leaking.

### Example 13

### In-Vivo Evaluation of Silk Coated Perivascular PU Films

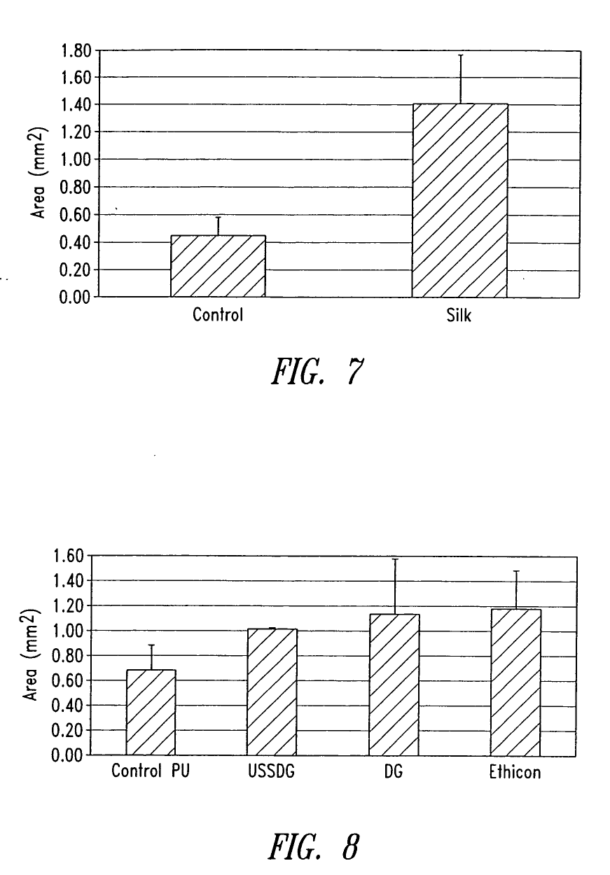

Wistar rats weighing 300 g to 400 g are anesthetized with halothane. The skin over the neck region is shaved and the skin is sterilized. A vertical incision is made over the trachea and the left carotid artery is exposed. A polyurethane film covered with silk strands or a control uncoated PU film is wrapped around a distal segment of the common carotid artery. The wound is closed and the animal is recovered. After 28 days, the rats are sacrificed with carbon dioxide and pressure-perfused at 100 mmHg with 10% buffered formaldehyde. Both carotid arteries are harvested and processed for histology. Serial cross-sections will be cut every 2 mm in the treated left carotid artery and at corresponding levels in the untreated right carotid artery. Sections are stained with H&E and Movat's stains to evaluate tissue growth around the carotid artery. Area of perivascular granulation tissue is quantified by computer-assisted morphometric analysis. Area of the granulation tissue is significantly higher in the silk coated group than in the control uncoated group. See FIG. 7.

### Example 14

### In-Vivo Evaluation of Perivascular PU Films Coated with Different Silk Suture Material

Wistar rats weighing 300 g to 400 g are anesthetized with halothane. The skin over the neck region is shaved and the skin is sterilized. A vertical incision is made over the trachea and the left carotid artery is exposed. A polyurethane film covered with silk sutures from one of three different manufacturers (3-0 Silk—Black Braided sutures from Davis & Geck, 3-0 silk sutures from US Surgical/Davis & Geck, sold under the tradename SOFSILK, and 3-0 Silk—Black Braided sutures from Ethicon Inc., sold under the tradename LIGAPAK) are wrapped around a distal segment of the common carotid artery. (The polyurethane film can also be coated with other agents that can induce fibrosis.) The wound is closed and the animal is recovered.

After 28 days, the rats are sacrificed with carbon dioxide and pressure-perfused at 100 mmHg with 10% buffered formaldehyde. Both carotid arteries are harvested and processed for histology. Serial cross-sections will be cut every 2 mm in the treated left carotid artery and at corresponding levels in the untreated right carotid artery. Sections are stained with H&E and Movat's stains to evaluate tissue growth around the carotid artery. Area of perivascular granulation tissue is quantified by computer-assisted morphometric analysis. Thickness of the granulation tissue is approximately the same in the three groups showing that tissue proliferation around silk suture is independent of manufacturing processes. See FIG. 8.

### Example 15

### In-Vivo Evaluation of Perivascular Silk Powder

Wistar rats weighing 300 g to 400 g are anesthetized with halothane. The skin over the neck region is shaved and the skin is sterilized. A vertical incision is made over the trachea and the left carotid artery is exposed. Silk powder is sprinkled on the exposed artery that is then wrapped with a PU film. Natural silk powder or purified silk powder (without contaminant proteins) is used in different groups of animals. Carotids wrapped with PU films only are used as a control group. The wound is closed and the animal is recovered. After 28 days, the rats are sacrificed with carbon dioxide and pressure-perfused at 100 mm Hg with 10% buffered formaldehyde. Both carotid arteries are harvested and processed for histology. Serial cross-sections will be cut every 2 mm in the treated left carotid artery and at corresponding levels in the untreated right carotid artery. Sections are stained with H&E and Movat's stains to evaluate tissue growth around the carotid artery. Area of tunica intima, tunica media and perivascular granulation tissue is quantified by computer-assisted morphometric analysis.

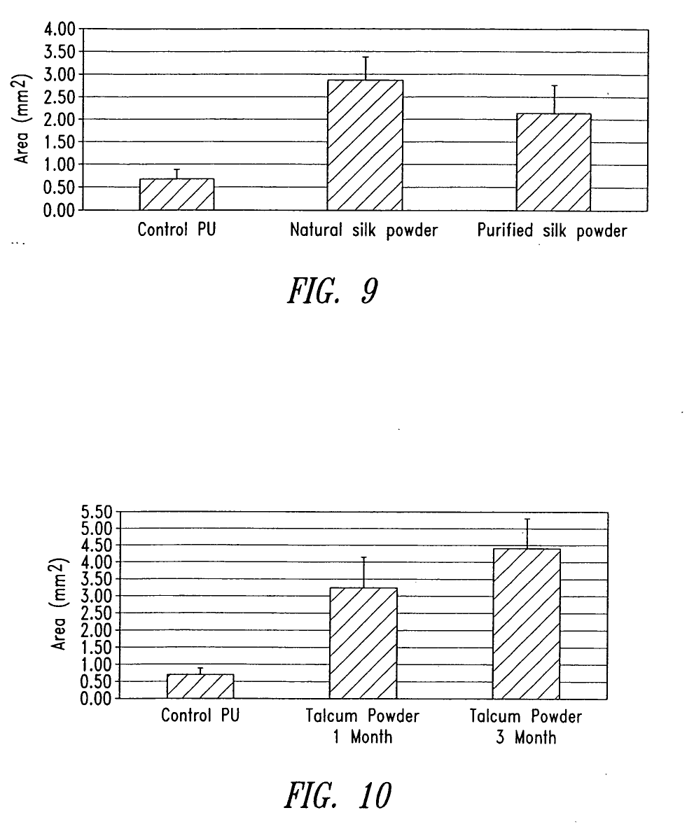

The natural silk caused a severe cellular inflammation consisting mainly of a neutrophil and lymphocyte infiltrate in a fibrin network without any extracellular matrix or blood vessels. In addition, the treated arteries were seriously damaged with hypocellular media, fragmented elastic laminae and thick intimal hyperplasia. Intimal hyperplasia contained many inflammatory cells and was occlusive in 2/6 cases. This severe immune response was likely triggered by antigenic proteins coating the silk protein in this formulation. On the other end, the regenerated silk powder triggered only a mild foreign body response surrounding the treated artery. This tissue response was characterized by inflammatory cells in extracellular matrix, giant cells and blood vessels. The treated artery was intact. These results show that removing the coating proteins from natural silk prevents the immune response and promotes benign tissue growth. Degradation of the regenerated silk powder was underway in some histology sections indicating that the tissue response will likely mature and heal over time. See FIG. 9.

### Example 16

### In-Vivo Evaluation of Perivascular Talcom Powder

Wistar rats weighing 300 g to 400 g are anesthetized with halothane. The skin over the neck region is shaved and the skin is sterilized. A vertical incision is made over the trachea and the left carotid artery is exposed. Talcum powder is sprinkled on the exposed artery that is then wrapped with a PU film. Carotids wrapped with PU films only are used as a control group. The wound is closed and the animal is recovered. After 1 or 3 months, the rats are sacrificed with carbon dioxide and pressure-perfused at 100 mmHg with 10% buffered formaldehyde. Both carotid arteries are harvested and processed for histology. Serial cross-sections will be cut every 2 mm in the treated left carotid artery and at corresponding levels in the untreated right carotid artery. Sections are stained with H&E and Movat's stains to evaluate tissue growth around the carotid artery. Thickness of tunica intima, tunica media and perivascular granulation tissue is quantified by computer-assisted morphometric analysis. Histopathology results and morphometric analysis showed the same local response to talcum powder at 1 month and 3 months. A large tissue reaction trapped the talcum powder at the site of application around the blood vessel. This tissue was characterized by a large number of macrophages within a dense extracellular matrix with few neutrophiles, lymphocytes and blood vessels. The treated blood vessel appeared intact and unaffected by the treatment. Overall, this result showed that talcum powder induced a mild long-lasting fibrotic reaction that was subclinical in nature and did not harm any adjacent tissue. See FIG. 10.

### Example 17

### In-Vivo Evaluation of Silk Coated Stent-Grafts

Sheep are anesthetized with an IV injection of Penthota and maintained with halothane. The skin over the neck is prepared for sterile surgery. A vertical skin incision is made over the stemocleidomastoid muscle on one side of the neck. The common carotid artery and the external jugular will be exposed. A 2 cm long arteriotomy will be performed after clamping the artery. A segment of the vein will be excised. One end of the vein graft is sutured to the arteriotomy with an end-to-side anastomosis. The other end is closed with suture thus creating a saccular aneurysm. After release of the clamps, the wound is closed in layers and the animal will then be recovered.

Two weeks later, the animal is anesthetized as previously described. Using sterile surgical technique, the right femoral artery is exposed and a vascular sheath inserted. A catheter is advanced through the sheath and guided by fluoroscopy into the carotid artery. A first angiogram of the aneurysm is performed. A DACRON stent-graft coated with silk strands or a control DACRON stent-graft without silk is inserted across the aneurysm thereby excluding it. A second angiogram is performed to check graft position. Catheter and sheath are removed. The femoral artery is repaired, the wound is closed and the animal is recovered.

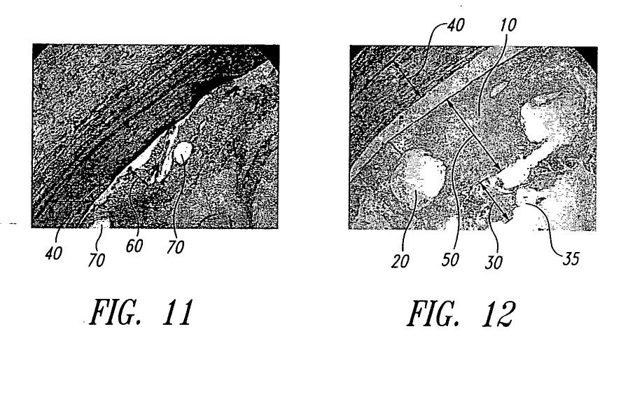

One month after stent-implantation, the animals are anesthetized as previously described. The left femoral artery is exposed and a vascular sheath inserted. A final angiogram is performed. The animal is then euthanized and pressure-perfused with formalin. The grafts and aneurysms are harvested, sectioned and stained with H&E and Movat's stains. Histopathology assessment of the stented arteries reveals that the space 10 between silk strands 20, stent graft 30 (where circular region 35 remains after removal of the stent tynes of stent graft 30) and vessel wall 40 is filled with tissue growth 50 (i.e., granulation tissue) which fills voids that are present after graft deployment and provides a tight seal (see, FIG. 12). In comparison, control grafts 60 without silk strands (shown in FIG. 11, where circular regions 70 remain after removal of the stent tynes of stent graft 60) exhibit no tissue growth between the graft 60 and the vessel wall 40.

All of the above U.S. patents, U.S. patent application publications, U.S. patent applications, foreign patents, foreign patent applications and non-patent publications referred to in this specification and/or listed in the Application Data Sheet are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A stent graft comprising an endoluminal stent and a graft, wherein the stent graft comprises silk.

2. The stent graft of claim 1 wherein the silk induces fibrosis between the stent graft and animal tissue.

3. The stent graft of claim 1 further comprising a biologically active agent, wherein the agent induces an enhanced fibrotic response in a host into which the stent graft has been inserted.

4. The stent graft of claim 1 wherein the silk is natural or recombinant silkworm silk or a derivative thereof.

5. The stent graft of claim 1 wherein the silk comprises fibroin.

6. The stent graft of claim 1 wherein the silk comprises sericin.

7. The stent graft of claim 1 wherein the silk is recombinant silk.

8. The stent graft of claim 1 wherein the silk is natural or recombinant spider silk or a derivative thereof.

9. The stent graft of claim 1 wherein the silk is in the form of a thread.

10. The stent graft of claim 1 wherein the silk is in the form of a braid.

11. The stent graft of claim 1 wherein the silk is in the form of a sheet.

12. The stent graft of claim 1 wherein the silk is in the form of powder.

13. The stent graft of claim 1 wherein the silk is acylated silk.

14. The stent graft of claim 1 wherein the silk is attached to the stent graft by interweaving the silk into the graft.

15. The stent graft of claim 1 wherein the silk is attached to the stent graft by means of an adhesive.

16. The stent graft of claim 1 wherein the silk is attached to the stent graft by means of suture.

17. The stent graft of claim 1 wherein the silk is attached only to the outside of the stent graft.

18. The stent graft of claim 1 wherein the silk is attached to distal regions of the stent graft.

19. The stent graft of claim 1 wherein a plurality of separated silk braids is attached to the stent graft.

20. The stent graft of claim 1 wherein the silk is attached to the stent portion of the stent graft.

21. The stent graft of claim 1 wherein the silk is attached to the graft portion of the stent graft.

22. The stent graft of claim 1 wherein the silk is present on the graft in an amount effective to induce a biological response in a host into which the stent graft has been inserted, where the biological response is a cellular matrix deposition between the stent graft and tissue adjacent to the stent graft.

23. The stent graft of claim 1 wherein the silk is present on the graft in an amount effective to induce a biological response in a host into which the stent graft has been inserted, where the biological response is an extracellular matrix deposition between the stent graft and tissue adjacent to the stent graft.

24. The stent graft of claim 1 further comprising a coating on some or all of the silk, where the coating degrades upon insertion of the stent graft into a host, the coating thereby delaying contact between the silk and the host.

25. The stent graft of claim 24 wherein the coating comprises a compound selected from the group consisting of gelatin, degradable polyesters, cellulose and cellulose derivatives, polysaccharides, lipids, fatty acids, sugar esters, nucleic acid esters, polyanhydrides, polyorthoesters and polyvinylalcohol.

26. The stent graft of claim 25 wherein the degradable polyester is selected from the group consisting of PLGA, PLA, MePEG-PLGA, PLGA-PEG-PLGA, and copolymers and blends thereof.

27. The stent graft of claim 25 wherein the cellulose derivative is hydroxypropyl cellulose.

28. The stent graft of claim 25 wherein the polysaccharide is selected from the group consisting of hyaluronic acid, dextran, dextran sulfate, and chitosan.

29. The stent graft of claim 1 wherein the silk induces fibrosis between the stent graft and animal tissue.

30. The stent graft of claim 1 wherein the silk induces adhesion between the stent graft and animal tissue.

31-69. (Canceled)

70. The stent graft of claim 1 wherein the stent graft is bifurcated.

71. The stent graft of claim 1 wherein the stent graft is a tube graft.

72. The stent graft of claim 1 wherein the stent graft is cylindrical.

73. The stent graft of claim 1 wherein the stent graft is self-expandable.

74. The stent graft of claim 1 wherein the stent graft is balloon-expandable.

75. The stent graft of claim 1 comprising distal ends, wherein the distal ends are adapted to release an agent that induces fibrosis.

76. The stent graft of claim 1 wherein the entire body of the stent graft is adapted to release an agent that induces fibrosis.

77. The stent graft of claim 1 wherein the stent graft is sterile.

78. The stent graft of claim 1 wherein the stent graft comprises an endoluminal stent and a graft, wherein the graft comprises an expandable portion that enhances the stiffness of the stent graft upon expansion.

79-99. (Canceled)

100. The stent graft of claim 2 further comprising a coating on some or all of the silk, where the coating degrades upon insertion of the stent graft into a host, the coating thereby delaying contact between the silk and the host.

101. The stent graft of claim 100 wherein the coating comprises a compound selected from the group consisting of gelatin, degradable polyesters, cellulose and cellulose derivatives, polysaccharides, lipids, fatty acids, sugar esters, nucleic acid esters, polyanhydrides, polyorthoesters and polyvinylalcohol.

102. The stent graft of claim 101 wherein the degradable polyester is selected from the group consisting of PLGA, PLA, MePEG-PLGA, PLGA-PEG-PLGA, and copolymers and blends thereof.

103. The stent graft of claim 100 wherein the cellulose derivative is hydroxypropyl cellulose.

104. The stent graft of claim 100 wherein the polysaccharide is selected from the group consisting of hyaluronic acid, dextran, dextran sulfate, and chitosan.

105. The stent graft of claim 2 wherein the silk induces adhesion between the stent graft and animal tissue.

106. The stent graft of claim 2 further comprising a biologically active agent, where the agent induces an enhanced fibrotic response in a host into which the stent graft has been inserted.

107. The stent graft of claim 106 wherein the agent is bleomycin or an analogue or derivative thereof.

108. The stent graft of claim 106 wherein the agent is selected from the group consisting of talcum powder, talc, ethanol, metallic beryllium and oxides thereof, silver nitrate, copper, silk, silica, crystalline silicates, and quartz dust.

109. The stent graft of claim 106 wherein the agent is selected from the group consisting of poly(ethylene-co-vinylacetate), polyurethane, and polymers and copolymers of acrylic acid.

110. The stent graft of claim 106 wherein the agent is vinyl chloride or a polymer of vinyl chloride.

111. The stent graft of claim 106 wherein the agent is is an adhesive selected from the group consisting of cyanoacrylate, crosslinked poly(ethylene glycol)—methylated collagen, and derivatives thereof.

112. The stent graft of claim 106 wherein the agent is selected from the group consisting of proteins, carbohydrates and peptides that contain cellular adhesion sequences.

113. The stent graft of claim 106 wherein the agent is an inflammatory cytokine.

114. The stent graft of claim 113 wherein the inflammatory cytokine is selected from the group consisting of TGFβ, PDGF, VEGF, aFGF , bFGF, TNFα, NGF, GM-CSF, IGF-a, IL-1, IL-8, IL-6, growth hormone, EDGF, CTGF, and peptide and non-peptide agonists, analogues and derivatives thereof.

115. The stent graft claim 106 wherein the agent is a component of extracellular matrix.

116. The stent graft of claim 115 wherein the component is vitronectin, fibronectin, chondroitin sulphate, laminin, hyaluronic acid, elastin, fibrin, fibrinogen, bitronectin, proteins found in basement membrane, fibrosin, or collagen.

117. The stent graft of claim 106 wherein the agent is selected from the group consisting of polylysine, chitosan, and N-carboxybutylchitosan.

118. The stent graft of claim 106 wherein the agent is a factor produced by immune cells.

119. The stent graft of claim 118 wherein the factor is selected from the group consisting of Interleukin-2 (IL-2), Interleukin-4 (IL-4), Interleukin-1 (IL-1), Interleukin-8 (IL-8), Interleukin-6 (IL-6) and peptide and non-peptide agonists, analogues and derivatives thereof.

120. The stent graft of claim 118 wherein the factor is selected from the group consisting of Granulocyte-Monocyte Colony-Stimulating-Factor (GM-CSM), monocyte chemotactic protein, histamine, and cell adhesion molecules.

121. The stent graft of claim 106 wherein the agent is selected from the group consisting of naturally occurring and synthetic peptides containing the RGD residue sequence.

122. The stent graft of claim 106 wherein the agent is a bone morphogenic protein (BMP).

123. The stent graft of claim 122 wherein the BMP is BMP-2, BMP-3, BMP-4, BMP-5, BMP-6, or BMP-7.

124. The stent graft of claim 106 wherein the agent is selected from the group consisting of inorganic and organic small anionic molecule stimulants.

125. The stent graft of claim 106 wherein the agent is wherein the agent is selected from the group consisting of DNA and RNA sequences which are capable of promoting synthesis of proteins that stimulate cell growth.

126. The stent graft of claim 2 further comprising a proliferative agent that stimulates cellular proliferation.

127. The stent graft of claim 126 wherein the proliferative agent is selected from the group consisting of dexamethasone, isotretinoin, 17-β-estradiol, diethylstibesterol, cyclosporin A, all-trans retinoic acid (ATRA), and analogues and derivatives thereof.

128. The stent graft of claim 2 further comprising a biologically active agent that inhibits or prevents expansion of an aneurysm.

129. The stent graft of claim 128 wherein the agent is a caspase inhibitor.

130. The stent graft of claim 129 wherein the caspase inhibitor is VX-799.

131. The stent graft of claim 128 wherein the agent is an MMP inhibitor.

132. The stent graft of claim 131 wherein the MMP inhibitor is BATIMASTAT or MARIMISTAT.

133. The stent graft of claim 128 wherein the agent is a tissue inhibitor of matrix metalloproteinases (TIMP).

134. The stent graft of claim 128 wherein the agent is a cytokine inhibitor.

135. The stent graft of claim 134 wherein the cytokine inhibitor is chlorpromazine, mycophenolic acid, rapamycin, or 1α-hydroxy vitamin D₃.

136. The stent graft of claim 128 wherein the agent is a MCP-1 antagonist.

137. The stent graft of claim 136 wherein the MCP-1 antagonist is nitronaproxen, Bindarit, or 1-alpha-25 dihydroxy vitamin D₃.

138. The stent graft of claim 128 wherein the agent is a TNFa antagonist or a TACE inhibitor.

139. The stent graft of claim 138 wherein the TACE inhibitor is E-5531, AZD-4717, glycophosphopeptical, UR-12715, cilomilast, infliximab, lentinan, or etanercept.

140. The stent graft of claim 128 wherein the agent is selected from the group consisting of IL-1, ICE, and IRAK antagonists.

141. The stent graft of claim 140 wherein the agent is E-5090, CH-172, CH-490, AMG-719, iguratimod, AV94-88, pralnacasan, ESONARIMOD, or tranexamic acid.

142. The stent graft of claim 128 wherein the agent is a chemokine receptor antagonist.

143. The stent graft of claim 142 wherein the chemokine receptor antagonist is ONO-4128, L-381, CT-112, AS-900004, SCH-C, ZK-811752, PD-172084, UK-427857, SB-380732, vMIP II, SB-265610, DPC-168, TAK-779, TAK-220, or KRH-1120.

144. The stent graft of claim 128 wherein the agent is an anti-inflammatory agent.

145. The stent graft of claim 144 wherein the anti-inflammatory agent is selected from the group consisting of dexamethasone, cortisone, fludrocortisone, prednisone, prednisolone, 6α-methylprednisolone, triamcinolone, betamethasone, and analogues and derivatives thereof.

146-425. (Canceled)

426. A method for treating a patient having an aneurysm, comprising delivering to a patient a stent graft of claim 1.

427. The method of claim 426 wherein the aneurysm is an abdominal aortic aneurysm.

428. The method of claim 426 wherein the aneurysm is a thoracic aortic aneurysm.

429. The method of claim 426 wherein the aneurysm is an iliac aortic aneurysm.

430. The method of claim 426 wherein the stent graft is delivered into a patient in a constrained form, and self-expands into place after release of a constraining device.

431. The method of claim 426 wherein the stent graft is delivered to the patient by balloon catheter.

432. A method for bypassing disease within a vessel, comprising delivering to a patient in need thereof a stent graft of claim 1, such that vessel contents bypass the diseased portion of the vessel.

433. The method of claim 432 wherein the stent graft is delivered into a patient in a constrained form, and self-expands into place after release of a constraining device.

434. The method of claim 432 wherein the stent graft is delivered to the patient by balloon catheter.

435. A method for creating communication between an artery and a vein, comprising delivering to a patient in need thereof a stent graft of claim 1, such that a passageway is created between the artery and vein.

436. The method of claim 435 wherein the stent graft is delivered into a patient in a constrained form, and self-expands into place after release of a constraining device.

437. The method of claim 435 wherein the stent graft is delivered to the patient by balloon catheter.

438. A method for creating communication between a first vein and a second vein, comprising delivering to a patient in need thereof a stent graft of claim 1, such that a passageway is created between the first and second veins.

439. The method of claim 438 wherein the stent graft is delivered into a patient in a constrained form, and self-expands into place after release of a constraining device.

440. The method of claim 438 wherein the stent graft is delivered to the patient by balloon catheter.

441. A method for reducing perigraft leakage associated with stent graft delivery in a patient, comprising delivering a stent graft of claim 1 to the patient.

442. The method of claim 441 wherein the stent graft is delivered into a patient in a constrained form, and self-expands into place after release of a constraining device.

443. The method of claim 441 wherein the stent graft is delivered to the patient by balloon catheter.

444. A method of adhering a stent graft in a patient in need thereof comprising inserting a stent graft of claim 1 into the patient.
